(19) United States
(12) Patent Application Publication
(10) Pub. No.: US 2021/0087618 A1
(43) Pub. Date: Mar. 25, 2021

(54) METHODS OF PERFORMING DIGITAL NUCLEIC ACID AMPLIFICATION USING POLYBUTENE

(71) Applicant: University of Washington, Seattle, WA (US)

(72) Inventors: Daniel T. CHIU, Seattle, WA (US); Jason E. KREUTZ, Marysville, WA (US)

(21) Appl. No.: 16/958,664

(22) PCT Filed: Jan. 22, 2019
(86) PCT No.: PCT/US2019/014355

§ 371 Date: Jun. 26, 2020

Related U.S. Application Data
(60) Provisional application No. 62/620,390, filed on Jan. 22, 2018.

(51) Int. Cl. C12Q 1/6851; C12Q 1/6804

(52) U.S. Cl. CPC C12Q 1/6851; C12Q 1/6804; C12Q 2527/125; C12Q 2565/629; C12Q 2531/113; C12Q 2563/107; C12Q 2565/601; C12Q 2527/146

(57) ABSTRACT

Methods, devices, and systems for performing digital assays are provided. In certain aspects, the digital assays comprise compartmentalized volumes. In certain aspects, the methods, devices, and systems can be used for the amplification and detection of nucleic acids. In certain aspects, the methods, devices, and systems can be used for the recognition, detection, and sizing of droplets in a volume.

### CROSS REFERENCE

This application claims the benefit of U.S. Provisional Patent Application No. 62/620,390, filed Jan. 22, 2018, the disclosure of which is herein incorporated by reference in its entirety.

### BACKGROUND OF THE INVENTION

Digital measurements are becoming increasingly more important in biology, owing to the robustness, higher sensitivity and higher accuracy that they offer. In addition, unlike analog measurements, where the measurement often must be calibrated with a running standard, digital measurements—based on counting of binary yes or no responses—do not require calibration and thus save user time, enhance robustness and ease of the assay.

### SUMMARY OF THE INVENTION

The present disclosure provides methods, devices and systems for performing digital measurements. More specifically, the present disclosure relates to methods, devices and systems for performing digital measurements in compartmentalized volumes. In some aspects, the compartmentalized volumes are in contact with an oil phase comprising polybutene. In some aspects, the present disclosure provides methods and apparatus for digital assays, including but not limited to digital PCR, digital isothermal nucleic acid amplifications (e.g. digital NASBA and digital LAMP), digital protein amplification (e.g. digital ELISA), digital single-molecule measurements, and other forms of digital measurements.

In one aspect, the present disclosure provides a method for performing a digital assay, the method comprising: producing a plurality of compartmentalized volumes (wherein each compartmentalized volume comprises an aqueous solution; at least some of the compartmentalized volumes comprise a nucleic acid; and at least some of the compartmentalized volumes are contacted with an oil phase comprising polybutene); amplifying the nucleic acid to produce an amplified nucleic acid product; and analyzing at least some of the plurality of compartmentalized volumes to determine the presence or absence of the amplified nucleic acid product.

In another aspect, the present disclosure provides a method for determining the concentration of a nucleic acid in a sample, the method comprising: producing a plurality of compartmentalized volumes (wherein each compartmentalized volume comprises an aqueous solution; at least some of the compartmentalized volumes comprise the nucleic acid; and at least some of the compartmentalized volumes are contacted with an oil phase comprising polybutene); amplifying the nucleic acid to produce an amplified nucleic acid product; analyzing at least some of the plurality of compartmentalized volumes to determine the presence or absence of the amplified nucleic acid product; determining volumes of at least some of the compartmentalized volumes; and determining the concentration of the nucleic acid in the sample based on the presence or absence of the amplified nucleic acid product in the compartmentalized volumes and the volumes of the at least some compartmentalized volumes.

In yet another aspect, the present disclosure provides a method as described above, wherein the plurality of compartmentalized volumes are in the form of a plurality of droplets. In other aspects, the disclosure provides a method as described above, wherein the plurality of compartmentalized volumes are positioned in a well plate. In other aspects, the disclosure provides a method as described above, wherein the well plate is a multi-well plate. In certain aspects, the disclosure provides a method as described above, wherein the plurality of compartmentalized volumes are positioned in a microfluidic device. In some aspects, the disclosure provides a method of loading the microfluidic device in sequence with a first fluid, an aqueous solution, and a second oil phase comprising polybutene, such that after loading at least a portion of the plurality of fluidic harbors comprise the aqueous solution separated into the compartmentalized volumes and separated by the second oil phase. In specific aspects, the disclosure provides a method as described above, wherein the first fluid comprises polybutene. In some aspects, the microfluidic device comprises a material selected from polydimethylsiloxane (PDMS), thermoset polyester (TPE), polymethylmethacrylate (PMMA), polyurethane methacrylate, polyethylene, polyester (PET), polytetrafluoroethylene (PTFE), polychlorotrifluoroethylene (PCTFE), polycarbonate, parylene, polyvinyl chloride, fluoroethylpropylene, Lexan™, polystyrene, cyclic olefin copolymers (COC), cyclic olefin polymers (COP), polyurethane, polyurethane blended with polyacrylate, polyestercarbonate, polypropylene, polybutylene, polyacrylate, polycaprolactone, polyketone, polyphthalamide, cellulose acetate, polyacrylonitrile, polysulfone, an epoxy polymer, a thermoplastic, polyvinylidene fluoride, polyamide, polyimide, glass, quartz, silicon, a gallium arsenide, a silicon nitride, fused silica, ceramic, metal, or a combination thereof. In some aspects, the microfluidic device comprises COP. In some aspects, the microfluidic device comprises COC. In certain aspects, the microfluidic device comprises COP and COC.

In certain aspects, the disclosure provides a method as described above, wherein the plurality of compartmentalized volumes are provided as an emulsion of the aqueous solution and the oil phase. In some aspects, the aqueous solution comprises a surfactant. In some aspects, the surfactant is selected from the group consisting of a non-ionic surfactant, an ionic surfactant, a silicone-based surfactants, a fluorinated surfactant, sorbitan monostearate (Span 60), octylphenoxyethoxyethanol (Triton X-100), polyoxyethylenesorbitan monooleate (Tween 80), sorbitan monooleate (Span 80), and ABIL WE 09. In certain aspects, the surfactant comprises greater than 0.01% w/w of the compartmentalized volume, greater than 0.1% w/w of the compartmentalized volume, greater than 0.25% w/w of the compartmentalized volume, greater than 0.5% w/w of the compartmentalized volume, greater than 1% w/w of the compartmentalized volume, greater than 5% w/w of the compartmentalized volume, or greater than 10% w/w of the compartmentalized volume. In some aspects, the disclosure provides a method as described above, wherein the oil phase further comprises a mineral oil, a light mineral oil, a silicone oil, a fluorinated oil, a fluorinated fluid, a fluorinated alcohol, Fluorinert, a Tegosoft®, Tegosoft® DEC, or a combination thereof.

In some aspects, the disclosure provides a method as described above, wherein the plurality of compartmentalized volumes is produced by agitating the aqueous solution and oil phase. In some aspects, the disclosure provides a method as described above, wherein the plurality of compartmentalized volumes is produced by extrusion through a channel opening or aperture.

In some aspects, the disclosure provides a method as described above, wherein the plurality of compartmentalized volumes is produced using a microfluidic device, the microfluidic device comprising: a body having a center region and an outer edge, the body being configured for rotating about a central axis and further comprising (i) a fluid inlet port located in the center region of the body; (ii) a flow channel having a proximal end, a distal end, and a flow axis, the flow channel in fluidic communication with the fluid inlet port; (iii) a plurality of fluidic harbors in fluidic communication with the flow channel and offset from the flow axis; and (iv) a fluid outlet port in fluidic communication with the flow channel, wherein the fluid outlet port is located closer to the center region of the body than the distal end of the flow channel. In certain aspects the device is loaded with a first fluid, subsequently the device is loaded with the aqueous solution such that the at least a portion of the plurality of fluidic harbors contains the aqueous solution, subsequently the device is loaded with a second oil phase comprising polybutene, such that the plurality of fluidic harbors are not in aqueous communication with one another. In certain aspects, the body comprises a substantially disc shape. In some aspects, the device is loaded using centrifugation. In some aspects, the device is loaded using pressure filling. In some aspects, the pressure filling comprises providing a positive pressure. In some aspects, the pressure filling comprises providing a negative pressure. In some aspects, loading the device using pressure filling comprises providing a negative pressure to an outlet of the device. In certain embodiments, the pressure filling comprises providing a vacuum. In some aspects, loading the device using pressure filling comprises providing a vacuum to an outlet of the device.

In certain aspects, the disclosure provides a method as described above, wherein the plurality of compartmentalized volumes is produced using a microfluidic device, the microfluidic device comprising: (i) a body comprising a proximal body portion and a distal body portion, wherein the proximal body portion comprises a fluid inlet port, an inlet reservoir, and a fluid outlet port, and the distal body portion comprises a fluid outlet reservoir, wherein the distal body portion is away from the proximal body portion; the fluid inlet port is connected to the inlet reservoir; and the fluid outlet port is connected to the fluid outlet reservoir by at least one return channel; (ii) at least one flow channel comprising a length extending from the inlet reservoir in the proximal body portion to outlet reservoir in the distal body portion, a proximal end in fluidic communication with the fluid inlet reservoir, a distal end in fluidic communication with the fluid outlet reservoir, and a flow axis; and (iii) a plurality of compartmentalized volumes in fluidic communication with the at least one flow channel and offset from the flow axis, wherein at least some of the plurality of compartmentalized volumes are contacted with an oil phase comprising polybutene. In certain aspects, the body comprises a substantially rectangular shape. In some aspects, the device is loaded using centrifugation.

In certain aspects, the present disclosure provides a method as described above, wherein the plurality of compartmentalized volumes is produced using a microfluidic device, the microfluidic device comprising a body having a center region, an outer edge and a central axis, the body being configured for rotating about the central axis and further comprising (i) a fluid inlet port positioned in the center region of the body; (ii) a flow channel having a flow axis and an outermost region, wherein the outermost region of the flow channel is the region of the flow channel that is farthest from the center region, and wherein the flow channel is in fluidic communication with the fluid inlet port; (iii) a plurality of fluidic harbors in fluidic communication with the flow channel and offset from the flow axis; and (iv) a fluid outlet port in fluidic communication with the flow channel, wherein the distance from the center region to the fluid outlet port is smaller than the distance from the center region to the outermost region of the flow channel. In certain aspects the device is loaded with a first fluid; subsequently the device is loaded with the aqueous solution such that the at least a portion of the plurality of fluidic harbors contains the aqueous solution; subsequently the device is loaded with the oil phase comprising polybutene, such that the plurality of fluidic harbors are not in aqueous communication with one another. In some aspects, the body comprises a substantially rectangular shape. In other aspects, the body comprises a substantially disc shape. In certain embodiments, the device is loaded using centrifugation.

In some aspects, the present disclosure provides a method as described above, wherein the aqueous solution further comprises a detectable agent for labeling the at least one nucleic acid sequence. In certain aspects, the detectable agent is fluorescent or luminescent. In some aspects, the detectable agent is selected from the group consisting of SYBR® green, Evagreen®, SYTO™-9, SYTO™-82, fluorescein, FITC, FAM™, rhodamine, HEX™ VIC®, JOE™, TET™, TAMRA™, ROX™, TRITC, Texas Red®, GFP, phycoerythrin (PE), a cyanine, a cyanine derivative, Cy™3, Cy™3.5, Cy™5, Cy™5.5, PE-Cy™5, Calcein, a BODIPY®, an Alexa Fluor®, a DyLight®Fluor, an ATTO, a Quasar® dye, a Cal Fluor®, a TYE™, a Qdot, a Cy™ dye, a SYSTO, a derivative thereof, a semiconducting polymer, and a semiconducting polymer dot.

In certain aspects, the present disclosure provides a method described as above, wherein the amplifying comprises applying to the at least one compartmentalized volumes at least one temperature, wherein the at least one temperature is sufficient to amplify the nucleic acid. In some aspects, the at least one temperature is isothermal. In certain aspects, the at least one temperature comprises at least two temperatures, and wherein the at least two temperatures undergo thermal cycling. In some aspects, a variable temperature is applied to the device during the amplifying. In certain aspects, the temperature is cycled between a high temperature and a low temperature during the amplifying.

In another aspect, the present disclosure provides a method described as above, wherein the amplifying comprises polymerase chain reaction (PCR), rolling circle amplification (RCA), nucleic acid sequence based amplification (NASBA), loop-mediated amplification (LAMP), Recombinase Polymerase Amplification (RPA), Helicase-Dependent Amplification (HDA), or a combination thereof. In certain aspects, the concentration of the detectable agent is determined over a dynamic range of at least three orders of magnitude. In some aspects, the concentration of the detectable agent is determined over a dynamic range of at least six orders of magnitude.

In certain aspects, the present disclosure provides a method described as above, wherein the compartmentalized volumes have a volume from at least 1 femtoliters (fL) to not more than 100 nanoliters (nL), from at least 10 fL to not more than 10 nL, from at least 100 fL to not more than 1 nL, from at least 1 picoliters (pL) to not more than 100 nL, from at least 10 pL to not more than 10 nL, from at least 100 pL to not more than 10 nL, from at least 1 pL to not more than 10 nL, from at least 1 pL to not more than 1 nL, from at least 50 fL to not more than 500 pL, or from at least 100 fL to not more than 100 pL. In specific embodiments, the compartmentalized volumes have a volume from at least 1 pL to not more than 100 nL.

In yet another aspect, the present disclosure provides a microfluidic device for discretizing a fluidic sample, the device comprising: (i) a body comprising a proximal body portion and a distal body portion, wherein the proximal body portion comprises a fluid inlet port, an inlet reservoir, and a fluid outlet port, and the distal body portion comprises a fluid outlet reservoir, wherein the distal body portion is away from the proximal body portion; the fluid inlet port is connected to the inlet reservoir; and the fluid outlet port is connected to the fluid outlet reservoir by at least one return channel; (ii) at least one flow channel comprising a length extending from the inlet reservoir in the proximal body portion to outlet reservoir in the distal body portion, a proximal end in fluidic communication with the fluid inlet reservoir, a distal end in fluidic communication with the fluid outlet reservoir, and a flow axis; and (iii) a plurality of compartmentalized volumes in fluidic communication with the at least one flow channel and offset from the flow axis, wherein at least some of the plurality of compartmentalized volumes are contacted with an oil phase comprising polybutene. In certain aspects, the body comprises a substantially rectangular shape. In certain aspects, the device comprises a plurality of flow channels substantially parallel to each other with a flow direction from the proximal body portion to the distal body portion. In some aspects, the device is loaded using centrifugation.

In another aspect, the present disclosure provides a microfluidic device for discretizing a fluidic sample, the device comprising: a body having a center region, an outer edge and a central axis, the body being configured for rotating about the central axis and further comprising (i) a fluid inlet port positioned in the center region of the body; (ii) a flow channel having a flow axis and an outermost region, wherein the outermost region of the flow channel is the region of the flow channel that is farthest from the center region, and wherein the flow channel is in fluidic communication with the fluid inlet port; (iii) a plurality of fluidic harbors in fluidic communication with the flow channel and offset from the flow axis; and (iv) a fluid outlet port in fluidic communication with the flow channel, wherein the distance from the center region to the fluid outlet port is smaller than the distance from the center region to the outermost region of the flow channel. In certain aspects the device is loaded with a first fluid; subsequently the device is loaded with the aqueous solution such that the at least a portion of the plurality of fluidic harbors contains the aqueous solution; subsequently the device is loaded with the oil phase comprising polybutene, such that the plurality of fluidic harbors are not in aqueous communication with one another. In some aspects, the body comprises a substantially rectangular shape. In other aspects, the body comprises a substantially disc shape. In certain embodiments, the device is loaded using centrifugation.

In certain aspects, the present disclosure provides a microfluidic device for discretizing a fluidic sample as above, wherein the compartmentalized volumes have a volume from at least 1 femtoliters (fL) to not more than 100 nanoliters (nL), from at least 10 fL to not more than 10 nL, from at least 100 fL to not more than 1 nL, from at least 1 picoliters (pL) to not more than 100 nL, from at least 10 pL to not more than 10 nL, from at least 100 pL to not more than 10 nL, from at least 1 pL to not more than 10 nL, from at least 1 pL to not more than 1 nL, from at least 50 fL to not more than 500 pL, or from at least 100 fL to not more than 100 pL. In specific embodiments, the compartmentalized volumes have a volume from at least 1 pL to not more than 100 nL.

In some aspects, the present disclosure provides a system for discretizing and analyzing fluidic samples, the system comprising: a rotor assembly comprising a central axis and a plurality of receptacles arranged radially around the central axis, each receptacle being shaped to receive a microfluidic device as in any one of the devices as described above, such that the center region of the body of the microfluidic device is positioned near the central axis and the outer edge of the body of the microfluidic device is positioned away from the central axis; a rotary actuator coupled to the rotor assembly; and one or more processors configured with instructions to cause the system to rotate the rotor assembly around the central axis using the rotary actuator.

In another aspect, the present disclosure provides a method of introducing a fluid into a microfluidic device, the method comprising: obtaining the microfluidic device of any one of devices as described above; and introducing a first fluid into the flow channel of the microfluidic device, wherein the first fluid comprises polybutene. In some aspects, the first fluid is introduced into the flow channel by centrifugal force driven flow.

In some aspects, the present disclosure provides a method as disclosed above, wherein the amplifying of the nucleic acid is compatible with polybutene. In certain aspects, being compatible is determined by (i) the ability to detect the amplified nucleic acid product; and (ii) analyzing the resulting amplification has less than 20% variance from the true value. In some aspects, the detectable agent is compatible with polybutene and nucleic acid amplification. In certain aspects, the amplifying comprises digital PCR (dPCR), digital LAMP (dLAMP), or a combination thereof.

In some aspects, the present disclosure provides a method as disclosed above, wherein at least some of the compartmentalized volumes comprise a nucleic acid and a protein. In some aspects, the nucleic acid is conjugated to the protein. In some aspects, the present disclosure provides a method as disclosed above, wherein at least some of the compartmentalized volumes comprise a nucleic acid and an antibody. In some aspects, the nucleic acid is conjugated to the antibody. In some aspects, at least some of the compartmentalized volumes comprise a target protein.

In some aspects, the present disclosure provides a microfluidic device for discretizing a fluidic sample, the device comprising: an inlet port; at least one flow channel having a flow axis, the at least one flow channel in fluidic communication with the inlet port; a plurality of compartmentalized volumes in fluidic communication with the at least one flow channel and offset from the flow axis, wherein at least some of the plurality of compartmentalized volumes are contacted with an oil phase comprising polybutene; and an outlet port in fluidic communication with the flow channel. In some aspects, the microfluidic device further comprises an inlet reservoir connected to the inlet port. In some aspects, the microfluidic device further comprises a fluid outlet reservoir connected to the fluid outlet port by at least one return channel. In some aspects, the compartmentalized volumes have a volume from at least 1 femtoliters (fL) to not more than 100 nanoliters (nL), from at least 10 fL to not more than 10 nL, from at least 100 fL to not more than 1 nL, from at least 1 picoliter (pL) to not more than 100 nL, from at least 10 pL to not more than 10 nL, from at least 100 pL to not more than 10 nL, from at least 1 pL to not more than 10 nL, from at least 1 pL to not more than 1 nL, from at least 50 fL to not more than 500 pL, or from at least 100 fL to not more than 100 pL. In some aspects, the compartmentalized volumes have a volume from at least 1 pL to not more than 100 nL.

In some aspects, the present disclosure provides a method of introducing a fluid into a microfluidic device, the method comprising: (i) obtaining the microfluidic device comprising: an inlet port; at least one flow channel having a flow axis, the at least one flow channel in fluidic communication with the inlet port; a plurality of compartmentalized volumes in fluidic communication with the at least one flow channel and offset from the flow axis, wherein at least some of the plurality of compartmentalized volumes are contacted with an oil phase comprising polybutene; and an outlet port in fluidic communication with the flow channel; and (ii) introducing a first fluid into the flow channel of the microfluidic device, wherein the first fluid comprises polybutene. In some aspects, the method further comprises introducing a fluid into the flow channel by pressure filling. In some aspects, the pressure filling comprises providing a positive pressure to the inlet port. In some aspects, loading the device using pressure filling comprises providing a negative pressure to the outlet port. In some aspects, loading the device using pressure filling comprises providing a vacuum to the outlet port.

In some aspects, the present disclosure provides a system for discretizing and analyzing fluidic samples, the system comprising: (i) an assembly comprising a plurality of receptacles, each receptacle being shaped to receive a microfluidic device, the microfluidic device comprising: an inlet port; at least one flow channel having a flow axis, the at least one flow channel in fluidic communication with the inlet port; a plurality of compartmentalized volumes in fluidic communication with the at least one flow channel and offset from the flow axis, wherein at least some of the plurality of compartmentalized volumes are contacted with an oil phase comprising polybutene; and an outlet port in fluidic communication with the flow channel; (ii) a pressurizing device connected to the microfluidic device; (iii) one or more processors configured with instructions to cause the system to provide a pressure to the microfluidic device; (iv) an optical detection component configured to optically analyze at least one of the compartmentalized volumes of the microfluidic device; and (v) a processing unit configured for controlling the pressuring device and the optical detection component, and configured for storing data generated from the optical detection component. In some aspects, the pressurizing device is attached to the inlet port of the microfluidic device, and the one or more processors are configured with instructions to cause the system to provide a positive pressure to the microfluidic device. In some aspects, the pressurizing device is attached to the outlet port of the microfluidic device, and the one or more processors are configured with instructions to cause the system to provide a negative pressure to the microfluidic device. In certain aspects, the pressurizing device is a vacuum.

For a fuller understanding of the nature and advantages of the present disclosure, reference should be had to the ensuing detailed description taken in conjunction with the accompanying drawings. The drawings represent embodiments of the present disclosure by way of illustration. The present disclosure is capable of modification in various respects without departing from the present disclosure. Accordingly, the drawings/figures and description of these embodiments are illustrative in nature, and not restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 depicts a microfluidic device for self-digitization of a fluidic sample.

FIG. 2A depicts samples prepared using a SD chip platform, wherein each plate was first flushed with an oil phase, then loaded with an aqueous phase, followed with the oil phase.

FIG. 2B depicts individual packets imaged during thermal cycling.

FIG. 2C depicts the number of positive wells and shows an agreement within the standard Poisson distribution confidence interval.

FIG. 3 depicts cyclic olefin polymer (COP) in contact with mineral oil or polybutene, prior to and following thermal cycling.

FIG. 4A depicts parallel digitization of a solution comprising Evagreen® Fluorophore under PCR conditions using an oil phase comprising polybutene.

FIG. 4B depicts parallel digitization of a solution comprising HEX™ Fluorophore-labeled DNA under PCR conditions using an oil phase comprising polybutene.

FIG. 4C depicts parallel digitization of a solution comprising Cy™5.5 Fluorophore-labeled DNA under PCR conditions using an oil phase comprising polybutene.

FIG. 4D depicts a composite image of the channels from FIGS. 4A-4C.

FIG. 5 depicts a rotor assembly of a system for self-digitization of fluidic samples.

FIG. 6A is a schematic diagram showing the individual components of a fully assembled chip.

FIG. 6B is an example layout of the microfluidic network.

FIG. 6C shows an example geometry of the side chamber array and main channel. All dimensions are in micrometers.

FIG. 7A depicts a configuration for SD devices spun using a bench-top centrifuge with a custom rotor designed for discs.

FIGS. 7B-7E depict a time series of a device (FIG. 7B) at the beginning, (FIGS. 7C and 7D) during, and (FIG. 7E) after loading.

FIG. 8 depicts a device for loading several arrays using centrifugal force.

FIG. 9A depicts samples analyzed using a SD chip platform following amplification, imaged via a FITC channel.

FIG. 9B depicts samples analyzed using a SD chip platform following amplification, imaged via a Cy™5 channel and a FITC channel.

FIG. 10A depicts samples in a COP device after amplification, imaged in a Cy™5 channel.

FIG. 10B depicts a linescan showing intensity across a number of compartments.

### DETAILED DESCRIPTION OF THE INVENTION

The present disclosure relates to methods, systems, and devices for performing digital assays. More specifically, the present disclosure relates to methods, systems, and devices for performing digital assays in compartmentalized volumes using an aqueous phase comprising one or more analytes and an oil phase comprising polybutene.

An important application for digital assays is the accurate quantification of DNA or RNA that is present in a sample. Here, the most widely used method to detect DNA or RNA is the polymerase chain reaction (PCR), where the sample is usually cycled between two or three temperatures around 60° C. and 95° C. The use of PCR to amplify DNA or RNA has greatly advanced a wide range of disciplines, ranging from basic biology to clinical diagnostics and forensics. One particular form of PCR that is often used in diagnostics and biomedical research is quantitative PCR (qPCR), which not only detects the presence of DNA or RNA in the sample, but also provides an accurate measure of its concentration. This is an important data point for making subsequent decisions and analysis—for example, the amount of a HIV therapeutic drug that is given to the patient, is determined by the amount of detected viral RNA load in the test sample.

Digital or limiting dilution DNA amplification has been developed, which can quantify the absolute number of template copies in the sample more accurately. In dPCR, the total sample is divided into an array of small volumes, such that, based on Poisson statistics, only few volumes contain one or more target molecules, while the majority of volumes contains no DNA. DNA amplification is then carried out in all volumes simultaneously and results in an increase of fluorescence in only those few volumes that contain target molecules. The DNA copy number is easily and accurately determined by counting the number of fluorescent volumes (i.e. those that contain a copy of DNA).

The concept of dPCR is appealing, but it was not widely used when the concept was first introduced because it could be difficult to create a large array of very small volumes (picoliters to nanoliters) used for dPCR. Additionally, widespread use of the method can also be impeded by precise temperature control and temperature cycling requirements. Generally, the temperature for the annealing and melting step is controlled within +/−1 degree Celsius. For many applications, where absolute quantification of DNA and RNA is important, these factors are difficult to meet or expensive to realize, in particular in resource-limited settings and at the point-of-care. To provide more ergonomic ways to amplify DNA and RNA in these settings, several isothermal methods have been developed, including rolling circle amplification (RCA), nucleic acid sequence based amplification (NASBA) or loop-mediated amplification (LAMP).

LAMP is an isothermal process for amplifying DNA or RNA with very high specificity at a fixed temperature between 60-65 degrees Celsius. Due to its high specificity it is able to discriminate single nucleotide differences during amplification. As a result, LAMP has been applied for SNP (single nucleotide polymorphism) typing. LAMP has also been shown to detect viral RNA with about ten-fold higher sensitivity than RT-PCR. Another feature, which differentiates LAMP from other isothermal methods, is the ability to directly correlate the amplification of DNA with the production of magnesium pyrophosphate, which increases the turbidity of the solution. The progress of the LAMP solution can thus be followed with a simple turbidimeter. Therefore, a non-homogeneous assay can be used for detecting the amplification products that result from LAMP. The production of magnesium pyrophosphate can also be used in form of a fluorescent indicator, which is particularly useful for digital assay readout. Before the reaction, a small amount of Calcein is added to the reaction mix. During amplification, the increased production of pyrophosphate leads to a sharp increase in Calcein fluorescence in those volumes that contain one or more target molecules. Furthermore, it is possible to perform colorimetric LAMP, for example, by using a pH sensitive probe, which facilitate equipment-free point-of-care usage of digital LAMP.

These reactions proceed at a fixed temperature, which reduces instrument complexity and lowers energy consumption, making them more suitable for point-of-care diagnostics and home-medicine devices. Translation of these methods into a digital format is an important step towards a better and more accurate detection of pathogens at the point-of-care. Moreover, digital assays would also improve the accuracy of protein amplification based assays, such as ELISA (Enzyme-Linked-Immunoadsorbent-Assay) or any single molecule based assay, where the single molecule assay may or may not require amplification.

In view of the above, there is a need to provide improved methods and systems for performing digital measurements. In addition, there is also a need to provide additional techniques using digital measurements, such as digital LAMP. The present disclosure provides these needs and more.

In some aspects, the present disclosure discloses the surprising and unexpected compatibility of cyclic olefin polymer (COP) devices with oil phases comprising polybutene in digital nucleic acid amplification reactions. In some aspects, the COP devices were compatible with the combination of polybutene and fluorescent probes. In certain embodiments, the present disclosure relates to methods, systems, and devices for performing digital assays in compartmentalized volumes using an aqueous phase comprising one or more analytes and an oil phase comprising polybutene, wherein the devices comprise COP.

### Apparatus and Methods of Self-Digitization

In some embodiments, the present disclosure provides methods, systems, devices and apparatuses for the discretization (also referred to as digitization), manipulation, and analyses of sample volumes that is robust and versatile. In some aspects, a fluidic device can partition a sample by exploiting the interplay between fluidic forces, interfacial tension, channel geometry, and the final stability of the formed droplet, compartmentalized volume, and/or discretized volume. These compartmentalized volumes allow for isolation of samples and partitioning into a localized array that can subsequently be manipulated and analyzed.

The devices of the present disclosure can be filled using a variety of methods, e.g., with either or both centrifugal force or fluidic pressure. In some embodiments, the devices of the present disclosure can be filled using centrifugal force. In some embodiments, the devices of the present disclosure can be filled using pressure filling. In certain embodiments, the devices of the present disclosure can be filled using a positive pressure. As a non-limiting example, devices of the present disclosure can be filled by applying a positive pressure to an inlet port of the devices. In some embodiments, the devices of the present disclosure can be filled using a negative pressure. As a non-limiting example, the devices of the present disclosure can be filled by applying a negative pressure to an outlet port of the devices. In some embodiments, devices of the present disclosure can be filled using a vacuum. In specific embodiments, devices of the present disclosure can be filled by providing a vacuum to the outlet port of the devices.

Some embodiments of the present disclosure can include methods and apparatuses for the analysis of species that include, but are not limited to, chemicals, biochemicals, genetic materials, or biological cells, using fluidic lattices to form fluidic packets. Potential applications for embodiments of the disclosure include but are not limited to, polymerase chain reaction (PCR), nucleic acid sequence-based amplification (e.g., loop mediated isothermal amplification (LAMP) and nucleic acid sequence-based amplification (NASBA)), crystallization of proteins and small molecules, and the analysis of cells (e.g., rare cells or single cells) or biological particles (e.g., isolated mitochondria) present in biological fluids. In some embodiments, the devices, methods and systems of the present disclosure can be used for polymerase chain reaction (PCR), reverse transcriptase PCR (RT-PCR), relative RT-PCR, competitive RT-PCR, comparative RT-PCR, real-time polymerase chain reaction (qPCR), RT-PCR/qPCR combined technique (qRT-PCR), digital PCR (dPCR), ligase chain reaction (LCR), loop mediated amplification (LAMP) (RT-LAMP), helicase dependent amplification (HDA) (RT-HDA), recombinase polymerase amplification (RPA) (RT-RPA), and/or strand displacement amplification (SDA) (RT-SDA). In certain embodiments, the devices, methods and systems of the present disclosure can be used for nucleic acid sequence based amplification (NASBA), transcription mediated amplification (TMA), self-sustained sequence replication (3SR), and single primer isothermal amplification (SPIA). Other techniques that can be used include, e.g., signal mediated amplification of RNA technology (SMART), rolling circle amplification (RCA), hyper branched rolling circle amplification (HRCA), exponential amplification reaction (EXPAR), smart amplification (SmartAmp), isothermal and chimeric primer-initiated amplification of nucleic acids (ICANS), and multiple displacement amplification (MDA).

In some aspects, the present disclosure includes devices for sample volume discretization and manipulation that can be tailored to suit a large variety of different applications and analysis methods. The components of the device can include, e.g., a main, or flow, channel(s) that is/are lined with arrays of adjacent sample compartments. The terms “main channel” and “flow channel” are used interchangeably herein. In one embodiment, the sample compartments can be positioned on and connected to the side of the main channel, and can be referred to as a side-harbor design. In another embodiment, the sample compartments can be at the bottom or top of the main channel, and can be referred to as a bottom-harbor design. The dimensions of the main channel, and the sample compartments can be varied to define the volume of the discretized sample, the compartment locations and the overall array size. The sample compartments, or chambers, that are in fluidic communication with a flow channel are hereafter also referred to as “fluidic harbors.” These fluidic harbors can be offset from an axis of flow through the main channel and can be located along a channel that provides shelter from the flow in the channel so that fluidic packets can be formed in the harbors.

As used herein, the term “in fluidic communication with” (and variations thereof) refers to the existence of a fluid path between components, and neither implies nor excludes the existence of any intermediate structures or components, nor implies that a path is always open or available for fluid flow.

The terms “oil phase” and “oil” are used interchangeably herein. Non-limiting examples of an oil phase can include a polybutene, a mineral oil (e.g., light mineral oil, heavy mineral oil, white mineral oil), a silicone oil, a fluorinated oil or fluid (e.g., a fluorinated alcohol or Fluorinert), other commercially available materials (e.g., Tegosoft®), or a combination thereof.

In certain embodiments, the oil phase comprises polybutene. Various grades of polybutene can be used. In some embodiments, polybutene has an average molecular weight of between 150-4,000 Mn, of between 170-3,500 Mn, of between 200-500 Mn, of between 200-1,000 Mn, of between 200-1,500 Mn, of between 200-2,000 Mn, of between 200-3,000 Mn, or an average molecular weight of between 300-2,400 Mn. In particular embodiments, polybutene has an average molecular weight of between 300-2,400 Mn. In certain embodiments, polybutene further comprises isobutylene. In some embodiments, polybutene comprises an isobutylene content of less than 1%, less than 5%, less than 10%, less than 20%, less than 33%, less than 50%, less than 66%, or less than 75%. In some embodiments, polybutene comprises an isobutylene content of between 1% and 100%, of between 5% and 100%, of between 10% and 100%, of between 10% and 90%, of between 10% and 80%, of between 20% and 100%, of between 20% and 80%, of between 30% and 100%, of between 30% and 70%, or of between 40% and 100%.

In some embodiments, the oil phase comprising polybutene further comprises an additive. In certain embodiments, the oil phase comprising polybutene further comprises an anionic detergent, a cationic detergent, a non-ionic detergent, a zwitterionic detergent, a non-detergent sulfobetaine, a surfactant, a wetting agent, or a combination thereof. In some embodiments, the oil phase comprising polybutene further comprises a surfactant or a wetting agent, or a combination thereof. In certain embodiments, the oil phase comprising polybutene further comprises 1-octanesulfonic acid sodium salt, chenodeoxycholic acid, cholic acid, deoxycholic acid, docusate sodium salt, lithium dodecyl sulfate, N-lauroylsarcosine, sodium 1-decasulfonate, sodium cholate hydrate, sodium deoxycholate, sodium dodecyl sulfate, sodium glycohenodeoxycholate, sodium glycocholate hydrate, sodium glycodeoxycholate, sodium hexanesulfonate, sodium pentanesulfonate, sodium taurocholate hydrate, sodium taurodeoxycholate, sodium taurosodeoxycholate, alkyltrimethylammonium bromide, benzalkonium chloride, benzyldimethylhexadecylammonium chloride, dimethyldioctadecylammonium bromide, dodecyltrimethylammonium bromide, hexadecylpyridinium chloride, hexadecyltrimethylammonium bromide, tetradecyltrimethylammonium bromide, 1-oleoyl-rac-glycerol, Brij L23, Brij 010, decaethylene glycol monododecyl ether, Digitonin, ECOSURF EH-9, ECOSURF SA-9, Genapol x-080, Igepal CA-630, Kolliphor P 188, Kolliphor EL, Mexoxypolyethylene glycol 350, N,N-dimethyldodecylamine N-oxide, n-dodecyl beta-D-manoside, Nonidet P 40 substitute, Pluronic F-127, Pluronic F-68, polysorbate 20, polysorbate 80, saponin, Span 80, Span 85, TERGITOL, Thesit, Triton CG-110, Triton x-100, Tween 20, Tween 80, Tyloxapol, CHAPS, CHAPSO, SB3-10, SB3-12, SB3-14, SB3-16, a derivative thereof, a variation thereof, or a combination thereof. In certain embodiments, the oil phase comprising polybutene further comprises Tegosoft® DEC.

In some embodiments, the oil phase comprising polybutene further comprises an additive, wherein the additive comprises less than 50% v/v, less than 40% v/v, less than 33% v/v, less than 25% v/v, less than 20% v/v, less than 19% v/v, less than 18% v/v, less than 17% v/v, less than 16% v/v, less than 15% v/v, less than 14% v/v, less than 13% v/v, less than 12% v/v, less than 11% v/v, less than 10% v/v, less than 9% v/v, less than 8% v/v, less than 7% v/v, less than 6% v/v, less than 5% v/v, less than 4% v/v, less than 3% v/v, less than 2% v/v, or less than 1% of the composition. In certain embodiments, the oil phase comprising polybutene further comprises an additive, wherein the additive comprises 0.01%-25% v/v, 0.01%-20% v/v, 0.01%-15% v/v, 0.01%-10% v/v, 0.01%-5% v/v, 0.01%-2.5% v/v, 1%-25% v/v, 1%-20% v/v, or 5%-25% v/v of the composition. In some embodiments, the oil phase comprising polybutene further comprises an additive, wherein the additive content by v/v is between 1% and 100%, between 5% and 100%, between 10% and 100%, between 10% and 90%, between 10% and 80%, between 20% and 100%, between 20% and 80%, between 30% and 100%, between 30% and 70%, or between 40% and 100%.

In certain embodiments, the oil phase comprising polybutene further comprises Abil WE 09, wherein the Abil WE 09 content is between 0.01% and 25% v/v of the oil phase. In certain embodiments, the oil phase comprising polybutene further comprises Tegosoft® DEC, wherein the Tegosoft® DEC content is between 1% and 99.9% v/v of the oil phase. In certain embodiments, the oil phase comprising polybutene further comprises Tegosoft® DEC, wherein the Tegosoft® DEC content is less than 100% of the oil phase. In certain embodiments, the oil phase comprises polybutene and Tegosoft® DEC present in a ratio. In specific embodiments, the ratio of Tegosoft® DEC to polybutene can range from 1:10 to 10:1, from 1:9 to 9:1, from 1:8 to 8:1, from 1:7 to 7:1, from 1:6 to 6:1, from 1:5 to 5:1, from 1:4 to 4:1, from 1:3 to 3:1, or from 1:2 to 2:1.

In some embodiments, the oil phase comprising polybutene further comprises another oil. In certain embodiments, the oil phase comprising polybutene further comprises a mineral oil, a fluorinated oil, a silicone based oil, hexadecane, a vegetable oil, a polyalphaolefin, a poly alkenylene glycol, a polyol ester, a synthetic non-hydrocarbon oil, a fluorosilicone, or Tegosoft® DEC.

In certain embodiments, the oil phase provides outstanding wettability. In certain aspects, the oil phase should not have a high viscosity. If viscosity is too high, the drainage from some devices disclosed herein can be slowed, which may result in slower or inadequate filling of fluidic harbors, or may result in greater droplet generation which in turn could result in less digitized or stepwise filling. In some embodiments, the oil phase has a viscosity of less than 500 centipoise, less than 400 centipoise, less than 300 centipoise, less than 200 centipoise, less than 100 centipoise, less than 50 centipoise, less than 30 centipoise, less than 20 centipoise, less than 10 centipoise, or less than 5 centipoise. In some embodiments, the oil phase has a viscosity of between 1 and 100 centipoise, between 5 and 40 centipoise, between 6 and 30 centipoise, or between 10 and 20 centipoise.

### Devices and Apparatuses for Self-Digitization of Sample Volumes

In certain embodiments, the present disclosure includes devices and apparatuses that comprise a plurality of channels, fluidic harbors, and reservoirs. The devices of the present disclosure can include, e.g., reservoirs, which can include large chambers used to store oil or aqueous samples near at least one inlet or outlet. Reservoirs are optional depending on the design. The devices can also include multiple types of flow channels. For example, the inlet channel(s) is/are where various fluids and sample can be introduced onto the chip. Branching elements can also be used to distribute the fluids and to sample many sets of flow channels and fluidic harbors simultaneously. The main filling channels can deliver the sample to the fluidic harbors, and can come in direct contact with the fluidic harbors. In some embodiments, the main channel can have features (e.g., indents or protrusions), the fluidic harbors can have features (e.g. bevels) or both the main channel and fluidic harbors can have features to help direct fluid flow. The connection between the main channel and fluidic harbors can also vary depending on the shape, offset and orientation of the fluidic harbor relative to the main channel. Drainage channels can be smaller and connect from the fluidic harbor to the main filling channel to provide a path for oil to drain out during filling. Drainage channels are not always necessary and can have varying complexity, with one or more drainage junctions connected to a given fluidic harbor. Some embodiments with fluidic harbors on the sides of channels utilize drainage channels, while embodiments with fluidic harbors on the bottom/top of channels do not. Outlet channels are capable of delivering any excess sample or oil away from the fluidic harbors to the outlet or outlet reservoir. They can include “resistor” channels to help establish more uniform flow between main channels. They can either connect to the outlet (reservoir) jointly in a de-branching fashion or individually. In some embodiments, the fluidic harbors can be, e.g., connected to both the main channel and any drainage channels. Suitable devices can include those in PCT application PCT/US14/44167 and in PCT application PCT/US16/41369 which are fully incorporated herein by reference.

In some aspects, the fluidic harbors can also function to discretize samples via geometric differences between the fluidic harbors and the channels and because of positional differences between the fluidic harbors and the channels (e.g., the fluidic harbors can be offset from the channels).

In certain embodiments, one or more of the fluidic harbor dimensions is greater than a corresponding dimension in the main channel. In such embodiments, the differences between the fluidic harbor dimensions and the corresponding dimensions of the flow channel facilitate the expansion of an aqueous solution loaded on the device into the larger volume of the fluidic harbor. Without being bound by theory, it is believed that this expansion occurs spontaneously because the larger dimensions in the fluidic harbor lowers the interfacial energy between the two fluids relative to what they are in the main channel.

In certain embodiments comprising fluidic harbors above or below the flow channels, the vertical dimension of the fluidic harbors, or height is larger than height of the channel. For embodiments with fluidic harbors on the sides of the main channel, both the vertical and a lateral dimension of the fluidic harbor can be larger than the same flow channel dimensions.

The fluidic harbors can be positioned in many different orientations. In certain embodiments the fluidic harbors are connected to the top/bottom of the main channel. In other embodiments the fluidic harbors are connected to the side of the main channel. In certain embodiments the “long” axis of the fluidic harbor still runs parallel to the main axis, but the harbor is offset from the channel. (The “long” axis refers to the direction of the longest dimension of the fluidic harbor). In certain further embodiments, the ratio of the long axis to the short axis is between 1 and 5.

For embodiments with fluidic harbors on the top/bottom of the main channels, the vertical dimension can be increased. For embodiments with fluidic harbors on the sides of the main channel, both the vertical and a lateral dimension can increase. In certain embodiments, the fluidic harbors are located on just one side of the main channel. In other embodiments, the fluidic harbors can be located on two sides of the main channel. Having fluidic harbors on two sides of the main channel can apply to both side- and bottom-harbor designs, and in certain embodiments fluidic harbors can be positioned on three or four sides. Fluidic harbors can take on various geometries, including but not limited to shape where the cross section is circular, oval, square, rectangular, triangular, or has some other polygonal dimensions. The fluidic harbors can also have rounded or beveled corners and can be asymmetrical in shape.

The fluidic harbors can be positioned in many different orientations. In certain embodiments the fluidic harbors are connected to the top/bottom of the main channel. In other embodiments the fluidic harbors are connected to the side of the main channel. In certain embodiments the “long” axis of the fluidic harbor still runs parallel to the main axis, but the harbor is offset from the channel. (The “long” axis refers to the direction of the longest dimension of the fluidic harbor). In other embodiments the “long” axis of the harbor is perpendicular to the main channel. In other embodiments the “long” axis is positioned at some other angle relative to the main flow axis. A fluidic harbor can also be said to be offset from an axis of flow through the flow channel if a line drawn between the center of harbor and the centerline of the flow channel is longer than the shortest distance between a channel wall and its centerline.

In some embodiments, the main channel can have a constant rectangular cross section. In certain embodiments additional constrictive or expansive features in the flow channel can be used to facilitate transport of sample to fluidic harbors and to discretize samples within the harbors. Fluidic harbors and/or the flow channels can be designed to have various dimensions according to a desired application. In certain embodiments the fluidic harbor can overlap with the channel, in other embodiments the fluidic harbor can be flush with the channel wall, and in other embodiments the fluidic harbor can be connected to the channel by a protrusion. Alternatively, or in addition to these connections, indents in the channel can in effect recreate overlap with the channel or the use of a protrusion or a flush meeting of the channel and fluidic harbor, without adjusting the position of the fluidic harbor relative to the main axis of the channel. In certain embodiments these additional channel features (e.g., indents or protrusions) in the channel are used to redirect flow and/or to help isolate fluidic harbors. The indents and protrusions can have various shapes and sizes to suite particular performance requirements. In certain embodiments, such as side-harbor designs, the features can be on the same side of the channel as the connection with the fluidic harbor. In some embodiments, constrictive or expansive features can be located on the opposite side of the channel. In other embodiments, there can be features on other channel sides as well. In certain embodiments, such as in bottom-harbor designs, the constrictive or expansive features can be adjacent to the bottom harbors but in the plane of the channel.

To assist with improved loading, the present disclosure also includes a microfluidic device that can include, e.g., a body having a center region and an outer edge. The body can be configured for rotating about a central axis and can include a variety of components. The body can have a variety of shapes. For example, the body can comprise a disc configuration. The body can be substantially rectangular-shaped; the body can be substantially disc-shaped; the body can be substantially triangular-shaped; the body can be substantially rhombus-shaped; the body can be substantially oval-shaped; the body can be substantially crescent-shaped; the body can be substantially square-shaped; the body can be substantially hexagon-shaped; the body can be substantially trapezoid-shaped; the body can be substantially pentagon-shaped. The body can have an axis (e.g., a central axis) that can be placed in the body to allow for rotation around the axis.

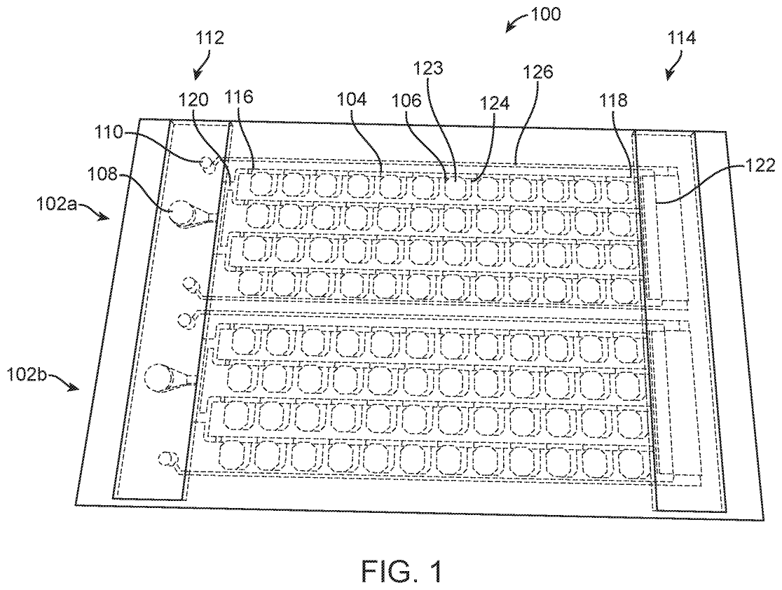

To assist with improved loading, the present disclosure includes another microfluidic device that can include, e.g., a body configured for rotating about a central axis and can include a variety of components. FIG. 1 illustrates a microfluidic device 100 for self-digitization of a fluidic sample, in accordance with embodiments. The device 100 includes one or more microfluidic arrays, e.g., a first microfluidic array 102a and a second microfluidic array 102b. In certain embodiments, different microfluidic arrays are used to discretize different samples and thus are not in fluidic communication with each other. In some embodiments, the microfluidic array (e.g., microfluidic array 102a) includes one or more flow channels 104, a plurality of fluidic harbors 106, one or more fluid inlet ports 108, and one or more fluid outlet ports 110. In some embodiments, the one or more flow channels 104 each have a length extending from a proximal portion 112 of the array to a distal portion 114 of the array. In some embodiments, the arrays of the microfluidic device 100 are filled by centrifugal loading, as discussed further herein, and the orientation of the array is defined relative to the axis of rotation such that “proximal” refers to the direction towards the axis of rotation and “distal” refers to the direction away from the axis of rotation. In such embodiments, centrifugation is used to drive fluid from the proximal portion 112 of the array to the distal portion 114 of the array.

In some embodiments, each flow channel 104 includes a proximal end 116 in fluidic communication with the one or more fluid inlet ports 108 and a distal end 118 in fluidic communication with the one or more fluid outlet ports 110. Optionally, in embodiments where a plurality of flow channels 104 are used, one or more branching channels 120 are used to couple the inlet port(s) 108 to the proximal end 116 of each flow channel 104. In some embodiments, the distal ends 118 of the flow channels 104 are each coupled to the outlet port(s) 110 via an outlet reservoir 122 configured to contain a relatively large volume of fluid (e.g., compared to the sample volumes of the fluidic harbors 106). In certain embodiments, the reservoir 122 is used to contain excess fluid during the filling procedure, as discussed further herein. Similarly, in some embodiments, an inlet reservoir is provided between the inlet port(s) 108 and the proximal ends 116 of the flow channels 104.

The fluidic harbors 106 are each coupled to the one or more flow channels 104 via a respective opening conduit 123. In some embodiments, the fluidic harbors are positioned along the length of a corresponding flow channel 104 between the proximal ends 116 and distal ends 118. Accordingly, a fluid sample loaded into the array via the outlet port(s) 110 is distributed into the fluidic harbors 106 via the flow channels 104 and is thereby discretized into individual sample volumes. In some embodiments, centrifugation is used to drive fluid into the fluidic harbors 106, as discussed further herein. Optionally, each fluidic harbor 106 also includes at least one drainage channel 124 coupling the fluidic harbor 106 to the flow channel 104. In some embodiments, drainage channels are used to control the filling rate of the fluidic harbors 106 and/or the completeness of filling.

In some embodiments, the inlet port(s) 108 and outlet port(s) 110 are both situated at or near the proximal array portion 112, with the reservoir 122 situated at the distal array portion 114. In such embodiments, one or more outlet return channels 126 extending from the distal portion 114 to the proximal portion 112 are used to fluidly couple the reservoir 122 to the outlet port(s) 108. In alternative embodiments, the inlet port(s) 108 are situated at the proximal portion 112 and the outlet port(s) 110 are situated at the distal portion 114, or vice-versa. The arrangement in which both the inlet port(s) 108 and outlet port(s) 110 are at the proximal portion 112 provides certain advantages for centrifugal loading. For instance, in some embodiments, having the outlet port(s) 110 at the proximal portion 112 near the axis of rotation causes flow rates to slow as the reservoir 122 fills up, as the fluid is unable to escape through the outlet(s) 110. This design feature provides a self-metering mechanism in which a set volume of fluid will pass through the fluidic harbor region before flow is automatically stopped. In various embodiments, this approach also reduces the likelihood of inadvertent leakage of the sample from the outlet port(s) 110 during centrifugation.

The design of the microfluidic devices described herein can be varied as desired. For instance, in some embodiments, a microfluidic device includes at least 100, at least 500, at least 1000, at least 5000, at least 10,000, at least 50,000, at least 100,000, at least 500,000 or at least 1 million fluidic harbors. In certain embodiments, each fluidic harbor has a volume of approximately 5 pL, 10 pL, 50 pL, 100 pL, 500 pL, 1 nL, 5 nL, 10 nL, 50 nL, 100 nL, or 500 nL, or within a range from about 5 pL to about 500 nL.

In some embodiments, the flow channels are arranged to extend parallel or substantially parallel to each other. In alternative embodiments, some or all of the flow channels do not extend parallel to each other. A flow channel can be linear, curved, or curvilinear, as desired. In some embodiments, a microfluidic array includes only parallel linear flow channels, and such arrays are referred to herein as “linear microfluidic arrays.” A flow channel can have a wide variety of cross-sectional shapes, such as a rectangular, trapezoidal, circular, semi-circular, oval, semi-oval, square, or triangular cross-sectional shape. In some embodiments, a flow channel has a single uniform cross-sectional shape throughout the length of the channel, while in other embodiments, the cross-sectional shape is variable along the length of the channel.

In some embodiments, the present disclosure provides a microfluidic device (e.g., a microfluidic chip) having one or more microfluidic arrays for discretizing one or more fluidic samples into a plurality of fluidic compartments. In certain embodiments, a microfluidic device includes 2, 4, 6, 8, 12, 16, 24, 32, 36, or 48 microfluidic arrays and/or at least 500, at least 1000, at least 5000, at least 10,000, at least 50,000, at least 100,000, at least 500,000, at least 1 million, at least 5 million, or at least 10 million fluidic harbors.

In some embodiments, the microfluidic devices of the present disclosure are configured for centrifugal loading of fluidic samples. In such embodiments, the microfluidic device includes a device body with a proximal body portion configured to be oriented towards the axis of rotation during centrifugation, and a distal body portion configured to be oriented away from the axis of rotation during centrifugation. Accordingly, the one or more microfluidic arrays are formed in the body of the microfluidic device with the flow channels (e.g., parallel flow channels) extending from the proximal body portion to the distal body portion, such that centrifugation drives fluid through the arrays from the proximal ends of the flow channels to the distal ends of the flow channels.

In some embodiments, the microfluidic devices of the present disclosure are configured for pressurized loading of fluidic samples. In such embodiments, the microfluidic device includes an inlet port and/or an outlet port. In some embodiment, the inlet port and/or outlet port can be attached to a pressurizing device. The pressurizing device can be a device that provides a positive pressure, a negative pressure, or a combination thereof. Non-limiting examples of pressurizing devices include pumps, positive displacement pumps, momentum transfer pumps, regenerative pumps, entrapment pumps, and vacuum pumps. Accordingly, the microfluidic devices are configured such that providing a pressure drives fluid through the flow channels of the devices, from the inlet port toward the outlet port.

In some embodiments, this disclosure provides a microfluidic device for discretizing a fluidic sample, the device comprising: (i) an inlet port; (ii) at least one flow channel having a flow axis, the at least one flow channel in fluidic communication with the inlet port; (iii) a plurality of compartmentalized volumes in fluidic communication with the at least one flow channel and offset from the flow axis, wherein at least some of the plurality of compartmentalized volumes are contacted with an oil phase comprising polybutene; and (iv) an outlet port in fluidic communication with the flow channel. In some embodiments, the device further comprises an inlet reservoir connected to the inlet port. In some embodiments, the microfluidic device further comprises a fluid outlet reservoir connected to the fluid outlet port by at least one return channel.

In some embodiments, this disclosure provides a method of introducing a fluid into a microfluidic device, wherein the method comprises obtaining the microfluidic device comprising: (i) an inlet port; (ii) at least one flow channel having a flow axis, the at least one flow channel in fluidic communication with the inlet port; (iii) a plurality of compartmentalized volumes in fluidic communication with the at least one flow channel and offset from the flow axis, wherein at least some of the plurality of compartmentalized volumes are contacted with an oil phase comprising polybutene; and (iv) an outlet port in fluidic communication with the flow channel. In some embodiments, the device further comprises an inlet reservoir connected to the inlet port; and introducing a first fluid into the flow channel of the microfluidic device, wherein the first fluid comprises polybutene. In some embodiments, the method comprises introducing a fluid into the flow channel by pressure filling. In specific embodiments, the pressure filling comprises providing a positive pressure to the inlet port. In other embodiments, loading the device using pressure filling comprises providing a negative pressure to the outlet port. In some embodiments, loading the device using pressure filling comprises providing a vacuum to the outlet port.

In some embodiments, this disclosure provides a system for discretizing and analyzing fluidic samples comprising: (a) an assembly comprising a plurality of receptacles, each receptacle being shaped to receive a microfluidic device, wherein the microfluidic device comprises: (i) an inlet port; (ii) at least one flow channel having a flow axis, the at least one flow channel in fluidic communication with the inlet port; (iii) a plurality of compartmentalized volumes in fluidic communication with the at least one flow channel and offset from the flow axis, wherein at least some of the plurality of compartmentalized volumes are contacted with an oil phase comprising polybutene; and (iv) an outlet port in fluidic communication with the flow channel; (b) a pressurizing device connected to the microfluidic device; (c) one or more processors configured with instructions to cause the system to provide a pressure to the microfluidic device; (d) an optical detection component configured to optically analyze at least one of the compartmentalized volumes of the microfluidic device; and (e) a processing unit configured for controlling the pressuring device and the optical detection component, and configured for storing data generated from the optical detection component. In some embodiments, the pressurizing device comprises a pump. In some embodiments, the pressurizing device comprises a vacuum pump. In certain embodiments, the pressurizing device is attached to the inlet port of the microfluidic device, and the one or more processors are configured with instructions to cause the system to provide a positive pressure to the microfluidic device. In other embodiments, the pressurizing device is attached to the outlet port of the microfluidic device, and the one or more processors are configured with instructions to cause the system to provide a negative pressure to the microfluidic device.

The microfluidic devices of the present disclosure can be designed in a variety of ways. For example, in some embodiments, the body of a microfluidic device comprises a substantially rectangular shape, a substantially square shape, a substantially circular shape, a substantially semi-circular shape, a substantially oval shape, a substantially semi-oval shape, a stadium shape, a squircle shape, or any other geometry. In certain embodiments, the body of a microfluidic device is shaped to be similar to existing devices and/or accommodate existing instrumentation, e.g., for convenience and compatibility. For instance, in various embodiments, the body is substantially rectangular with a length (e.g., along the proximal-distal direction) of approximately 127.8 mm and a width (e.g., orthogonal to the proximal-distal direction) of approximately 85.5 mm, similar to a 96-well microplate. As another example, in various embodiments, the body is substantially rectangular with a length of approximately 100 mm and a width of approximately 75 mm, similar to the adapter size for certain PCR thermal cycling devices. As used herein, substantially rectangular can refer to a rectangle, a rounded rectangle, or a beveled rectangle.

In some aspects, the microfluidic devices can include one or more fluid inlet ports. The fluid inlet ports can be located in the center region of the body. The devices can also include one or more flow channels. The flow channels can include a proximal end, a distal end, and a flow axis. The flow channels can also be in fluid communication with the fluid inlet port. In some embodiments, the proximal end of the flow channel is near or in the vicinity of the center region and the distal end is located near the outer edge of the body. The location of the fluid inlet ports in the center region of the body can be designed for centrifugal loading, e.g., such that upon spinning of the body fluid will be directed into the flow channels and towards the outer edge of the body. Alternatively, other methods for loading fluid in the channels can also be used, e.g., by pressure loading. In some embodiments, fluid can be loaded in the channels by using pressure filling. In certain embodiments, fluid can be loaded in the channels by using a positive pressure. As a non-limiting example, fluid can be loaded in the channels by applying a positive pressure to an inlet port of a device. In some embodiments, fluid can be loaded in the channels by using a negative pressure. As a non-limiting example, fluid can be loaded in the channels by applying a negative pressure to an outlet port of a device. In some embodiments, fluid can be loaded in the channels by using a vacuum. In specific embodiments, fluid can be loaded in the channels by providing a vacuum to the outlet port of a device. The devices can further include a plurality of fluidic harbors in fluid communication with the flow channel and offset from the flow axis. The fluidic harbors are explained in more detail below.

The microfluidic devices of the present disclosure can also include one or more fluid outlet ports. The fluid outlet ports can be in fluid communication with the flow channel. In some embodiments, the one or more fluid outlet ports can be located closer to the center region of the body than the distal end of the flow channel. The location of the fluid outlet ports can affect loading of the flow channels and fluidic harbors with fluid. In some embodiments, the outlet can be at the same radial position of the inlet, or closer to the exterior or edge of the device than the inlet, or even closer to the interior or center of the device than the inlet. Having the outlet located in the center region rather than near the outer edge of the body can provide, e.g., an additional level of control for loading the flow channels and fluidic harbors.

In some embodiments, the devices described herein can have a body that can have a shape and can be configured to be rotated for, e.g., filling of the channels in the devices. In some aspects, the central region and outer edge of the device can be defined according to a radius of the circular disc-shaped body. The disc-shaped body can have a radius extending from the center of the body to the body's outer edge. The center region of the body can be defined according to a position along the radius of the body. For example, the center region can be a circle within the circular disc body. In some embodiments, the center region can include the dead center of a disc-shaped body, e.g., the dead center of a circle. If the radius from the center of the disc body to the outer edge is a length of 1, then the center region can be defined according to a percentage or ratio of that radius of length 1. In certain aspects, the center region on the disc body can be defined by a circle having a radius of between about 0 to less than about 0.8, between about 0.01 to less than about 0.8, between about 0.01 to less than about 0.7, between about 0.01 to less than about 0.6, between about 0.01 to less than about 0.5, between about 0.01 to less than about 0.4, between about 0.01 to less than about 0.3, between about 0.01 to less than about 0.2, or between about 0.01 to less than about 0.1 of the radius of length 1. In some aspects, the center region on the disc body can be defined by a circle having a radius of between about 0.1 to about 0.40, between about 0.2 to about 0.3, between about 0.1 to about 0.3, or between about 0.1 to about 0.2 of the radius of length 1.

As provided herein, one or more fluid outlet ports in the devices can be located in the center region of the body. The fluid outlet ports can be positioned in the same radial position of the fluid inlet ports. For example, the fluid inlet and outlet ports can be positioned in the center region at radial positions of between about 0.05 to less than about 0.50 of the radius of length 1. In some aspects, the center region on the disc body can be defined by a circle having a radius of between about 0.1 to about 0.40, between about 0.2 to about 0.3, between about 0.1 to about 0.3, or between about 0.1 to about 0.2 of the radius of length 1. In some aspects, the device can include one inlet port that is located at the dead center of a device, e.g., a circular device. One or more outlet ports can be coupled to the inlet port and positioned at a variety of radial positions in the device. Alternatively, the fluid outlet ports can be positioned in the center region but at a different radial position of the fluid inlet ports. For instance, the fluid outlet ports can be located at a radial position of 0.1 along the full radius of length 1, and the fluid inlet ports can be located at a radial position of 0.2 along the full radius of length 1. In some embodiments, the fluid outlet ports can be located at a radial position of 0.2 along the full radius of length 1, and the fluid inlet ports can be located at a radial position of 0.1 along the full radius of length 1. In some embodiments, different fluid inlet and outlet ports can be positioned at different radial positions in the center region. For example, one fluid inlet port can be located at a radial position of 0.1 and another fluid inlet port can be located at a radial position of 0.2. One fluid outlet port can be located at a radial position of 0.15 and another fluid inlet port can be located at a radial position of 0.25.

In another aspect, the present disclosure can also include microfluidic devices that can include one or more micro-well plates. The micro-well plates can include a plurality of wells (e.g., 96 well plates) that can be designed to provide a fluidic system for, e.g., loading and analysis of samples in each well. In some aspects, the wells can include one or more fluid inlet ports, one or more flow channels, and one or more fluid outlet ports. Each of the components can be in fluidic communication. The wells can also include fluidic harbors that can, e.g., be arranged along one or more of the flow channels that take in samples from the wells.

The wells can be arranged on the devices or micro-well plates in a variety of ways. For example, the wells can be arranged in an array format to allow, e.g., for high throughput analysis of the fluidic harbors. In some embodiments, the wells can be arranged in a square matrix array or a rectangular matrix array having a 2:3 ratio or a 3:4 ratio of rows to columns. The microfluidic devices can include any number of wells that can adequately fit on a device, if desired. For example, the number of wells present can be, e.g., 6, 12, 24, 48, 96, 384 or 1536 wells. Other well configurations and numbers can be used.

In various aspects, the present disclosure provides microfluidic devices comprising: a body having a center region and an outer edge, the body being configured for rotating about a central axis and further comprising: a fluid inlet port positioned in the center region of the body; a flow channel having a flow axis and an outermost region, wherein the outermost region of the flow channel is the region of the flow channel that is farthest from the center region, and wherein the flow channel is in fluidic communication with the fluid inlet port; a plurality of fluidic harbors in fluidic communication with the flow channel and offset from the flow axis; and a fluid outlet port in fluidic communication with the flow channel, wherein the distance from the center region to the fluid outlet port is smaller than the distance from the center region to the outermost region of the flow channel.

In various aspects, the present disclosure provides microfluidic devices comprising: a body having a center region, an outer edge and a central axis, the body being configured for rotating about the central axis and further comprising: a fluid inlet port positioned in the center region of the body; a flow channel having a flow axis and an outermost region, wherein the outermost region of the flow channel is the region of the flow channel that is farthest from the center region, and wherein the flow channel is in fluidic communication with the fluid inlet port; a plurality of fluidic harbors in fluidic communication with the flow channel and offset from the flow axis; and a fluid outlet port in fluidic communication with the flow channel, wherein the distance from the center region to the fluid outlet port is smaller than the distance from the center region to the outermost region of the flow channel.

In some aspects, the microfluidic device further comprises a flow cell, wherein the flow cell comprises the fluid inlet port, the fluid outlet port, and a plurality of the flow channels, wherein each of the flow channels is in fluidic communication with the fluid inlet port and the fluid outlet port. In other aspects, the devices comprise a plurality of flow channels configured such that the flow axis of each flow channel is perpendicular to the outer edge of the body. In some aspects, the plurality of flow channels is configured such that the flow channels are arranged in parallel. In certain aspects, at least one of the fluidic harbors is at an angle other than orthogonal to the flow axis.

In some aspects, the device comprises a material selected from polydimethylsiloxane (PDMS), thermoset polyester (TPE), polymethylmethacrylate (PMMA), polyurethane methacrylate, polyethylene, polyester (PET), polytetrafluoroethylene (PTFE), polychlorotrifluoroethylene (PCTFE), polycarbonate, parylene, polyvinyl chloride, fluoroethylpropylene, Lexan™, polystyrene, cyclic olefin copolymers (COC), cyclic olefin polymers (COP), polyurethane, polyurethane blended with polyacrylate, polyestercarbonate, polypropylene, polybutylene, polyacrylate, polycaprolactone, polyketone, polyphthalamide, cellulose acetate, polyacrylonitrile, polysulfone, an epoxy polymer, a thermoplastic, polyvinylidene fluoride, polyamide, polyimide, glass, quartz, silicon, a gallium arsenide, a silicon nitride, fused silica, ceramic, metal, or a combination thereof. In specific embodiments, the device comprises COP. In certain embodiments, the device comprises COP. In certain embodiments, the device comprises COP and COC.

In certain aspects, at least one of the flow channels and the plurality of fluidic harbors comprise a hydrophobic surface. In other aspects, at least a portion of the body comprises natively hydrophobic or surface-treated polydimethylsiloxane (PDMS), polycarbonate (PC), glycol modified polyethylene terephthalate (PETG), cyclic olefin copolymer (COC), cyclic olefin polymer (COP), polychlorotrifluoroethylene (PCTFE), a multilaminate material, or a combination thereof. In certain embodiments, at least a portion of the body comprises COP. In some embodiments, at least a portion of the body comprises COC. In some embodiments, at least a portion of the body comprises COC and COP. In further aspects, at least one of the flow channel and the plurality of fluidic harbors comprise a fluorophilic surface.

In some aspects, the present disclosure provides microfluidic devices that are loaded with a first fluid. In various aspects, the first fluid comprises an oil phase. In further aspects, the first fluid comprises a polybutene, a fluorinated oil, a hydrocarbon oil, a silicone oil, or a combination thereof.

In some aspects, the microfluidic device further comprises a common fluid reservoir, wherein the common fluid reservoir is in fluidic communication with the distal end of each of the flow channels and wherein the common fluid reservoir is in fluidic communication with the fluid outlet port. In other aspects, the fluid outlet port is located closer to a center of the body than the fluid inlet port. In further aspects, the fluid outlet port is located farther from a center of the body than the fluid inlet port.

In yet further aspects, the fluid outlet port is located as close to a center of the device as the fluid inlet port.

In some aspects, the present disclosure provides microfluidic devices further comprising a plurality of flow cells, wherein each flow cell comprises: a plurality of flow channels; a fluid inlet port; and a fluid outlet port, wherein the fluid outlet port is located closer to the center region of the body than the distal ends of the flow channels.

In other aspects, the present disclosure provides a plurality of flow cells, wherein each flow cell comprises: a plurality of flow channels; a fluid inlet port; and a fluid outlet port, wherein the distance from the center region to the fluid outlet port is smaller than the distance from the center region to an outermost region of the flow channel.

In some aspects, each of the flow cells comprises a plurality of fluid inlet ports, a plurality of fluid outlet ports, or a combination thereof.

In various aspects, the present disclosure provides analytical systems comprising: a rotation component configured for rotating any microfluidic device of the present disclosure about its central axis; an optical detection component configured to optically analyze at least one of the fluidic harbors of the microfluidic device; and a processing unit configured for controlling the rotation component and the optical detection component, and configured for storing data generated from the optical detection component.

In certain aspects, the analytical systems further comprise a plurality of fluid reservoirs each capable of containing a fluid, wherein the plurality of fluid reservoirs is configured to provide the fluid to the fluid inlet port of the microfluidic device. In some aspects, the analytical systems further comprise a heat-control component configured to apply heat to the plurality of fluidic harbors.

In some aspects, the heat control component is configured to heat the plurality of fluidic harbors sufficiently to perform polymerase chain reaction (PCR), isothermal amplification, or a combination thereof.

In some aspects, the analytical systems further comprise a fluid introduction component configured to move a fluid through the flow channel. In certain aspects, the fluid introduction component comprises a source of pressure in fluidic communication with at least one of the fluid inlet ports, wherein the source of pressure is configured to apply positive or negative pressure sufficient to move a fluid through the flow channel, and wherein the positive or negative pressure is selected from a group consisting of: air pressure, pneumatic pressure, hydraulic pressure, or a combination thereof. In other aspects, the fluid introduction component is configured to move a fluid through the flow channel by means of capillary action, wicking, or centrifugal force driven flow.

In some aspects, the optical detection component is configured to analyze the microfluidic device rotating between 50 RPM and 2000 RPM. In certain aspects, the optical detection component comprises a microscope, a laser scanner, an optical disc drive, or a combination thereof.

In some aspects, the rotation component is configured to adjust a speed of rotation of the microfluidic device to match a readout speed of the optical detection component. In certain aspects, the rotation component is configured to rotate the device microfluidic between 50 RPM and 5000 RPM. In further aspects, the analytical systems further comprise an optical disc drive. In other aspects, the optical disc drive is a compact disc (CD) drive, a digital video disc (DVD) drive, a Blu-ray drive, or a modified version thereof, or a combination thereof.

In some aspects, the analytical systems are configured for housing, rotating, and processing a plurality of the microfluidic devices.

In various aspects, the present disclosure provides microfluidic devices comprising: a multi-well plate comprising a plurality of wells, wherein each of the wells further comprises: a fluid inlet port in fluidic communication with the well; a fluid outlet port in fluidic communication with the well; a flow channel having a flow axis, the flow channel in fluidic communication with the fluid inlet port and the fluid outlet port; and a plurality of fluidic harbors in fluidic communication with the flow channel and offset from the flow axis.

In some aspects, at least one of the wells comprises a plurality of the flow channels. In other aspects, at least one of the wells comprises a plurality of the fluid inlet ports. In further aspects, at least one of the wells comprises a plurality of the fluid outlet ports. In certain aspects, the plurality of wells is arranged in an array. In further aspects, the plurality of wells is arranged in square matrix array. In yet further aspects, the plurality of wells is arranged in a rectangular matrix array having a 2:3 ratio or a 3:4 ratio of rows to columns. In other aspects, the microfluidic device contains 6, 12, 24, 48, 96, 384 or 1536 wells. In some aspects, at least one of the fluidic harbors is at an angle other than orthogonal to the flow axis. In other aspects, at least one of the fluidic harbors is at an angle orthogonal to the flow axis. In further aspects, each of the fluidic harbors is in fluidic communication with the flow channel by an opening conduit. In some aspects, at least one of the fluidic harbors comprises a channel in fluidic communication with the flow channel.

In some aspects, at least one of the flow channel and the fluidic harbor comprises a hydrophobic surface. In certain aspects, the microfluidic devices of the present disclosure comprise a material selected from polydimethylsiloxane (PDMS), thermoset polyester (TPE), polymethylmethacrylate (PMMA), polyurethane methacrylate, polyethylene, polyester (PET), polytetrafluoroethylene (PTFE), polychlorotrifluoroethylene (PCTFE), polycarbonate, parylene, polyvinyl chloride, fluoroethylpropylene, Lexan™, polystyrene, cyclic olefin copolymers (COC), cyclic olefin polymers (COP), polyurethane, polyurethane blended with polyacrylate, polyestercarbonate, polypropylene, polybutylene, polyacrylate, polycaprolactone, polyketone, polyphthalamide, cellulose acetate, polyacrylonitrile, polysulfone, an epoxy polymer, a thermoplastic, polyvinylidene fluoride, polyamide, polyimide, glass, quartz, silicon, a gallium arsenide, a silicon nitride, fused silica, ceramic, metal, or a combination thereof. In some embodiments, the microfluidic devices of the present disclosure comprise COP. In some embodiments, the microfluidic devices of the present disclosure comprise COC. In some embodiments, the microfluidic devices of the present disclosure comprise COC and COP.

In certain aspects, each well further comprises an inner chamber and an outer chamber, wherein the inner chamber is in fluidic communication with the inlet port and the outer chamber is in fluidic communication with the outlet port. In further aspects, the configuration of the inner chamber and the outer chamber is sufficient to create a non-circular direction of flow through the flow channel.

In some aspects, at least a portion of the device comprises natively hydrophobic or surface treated polydimethylsiloxane (PDMS), polycarbonate (PC), glycol modified polyethylene terephtalate (PETG), cyclic olefin copolymer (COC), cyclic olefin polymer (COP), polychlorotrifluoroethylene (PCTFE), or multilaminate materials to provide a hydrophobic surface for the flow channel, the plurality of fluidic harbors, or a combination thereof. In some embodiments, at least a portion of the device comprises COC. In some embodiments, at least a portion of the device comprises COP. In some embodiments, at least a portion of the device comprises COP and COC.

In certain aspects, the device is loaded with a first fluid, the first fluid comprising an oil phase. In further aspects, the first fluid comprises a polybutene, a fluorinated oil, a hydrocarbon oil, a silicone oil, or a combination thereof. In certain aspects, the device is loaded with a polybutene.

### Materials for Self-Digitization of Sample Volumes

In certain embodiments, the devices of the present disclosure can be composed of a material with suitable surface properties to facilitate the digitization process. For example, devices of the present disclosure can be fabricated from polydimethylsiloxane (PDMS) or/and glass. Other substrate materials can include but are not limited to silicon, thermoset polyester (TPE), polymethylmethacrylate (PMMA), polyurethane methacrylate, polyethylene, polyester (PET), polytetrafluoroethylene (PTFE), polychlorotrifluoroethylene (PCTFE), polycarbonate, parylene, polyvinyl chloride, fluoroethylpropylene, Lexan™, polystyrene, cyclic olefin copolymers (COC), cyclic olefin polymers (COP), polyurethane, polyurethane blended with polyacrylate, polyestercarbonate, polypropylene, polybutylene, polyacrylate, polycaprolactone, polyketone, polyphthalamide, cellulose acetate, polyacrylonitrile, polysulfone, epoxy polymers, thermoplastics, fluoropolymer, and polyvinylidene fluoride, polyamide, polyimide, inorganic materials (glass, quartz, silicon, gallium arsenides, silicon nitride), fused silica, ceramic, glass (organic), metals and/or other materials and combinations thereof. In some embodiments, the devices of the present disclosure can be fabricated from COC. In some embodiments, the devices of the present disclosure can be fabricated from COP. In certain embodiments, the devices of the present disclosure can be fabricated using COC. In some embodiments, the devices of the present disclosure can be fabricated using COP. In some embodiments, the devices of the present disclosure can be fabricated using COP and COC.

For biological assays, a device can be based on a polymer material so the device is disposable for one-time use. In some embodiments, a portion of a device of the present disclosure (e.g., the body) can include natively hydrophobic or surface treated polydimethylsiloxane (PDMS), polycarbonate (PC), glycol modified polyethylene terephthalate (PETG), cyclic olefin copolymer (COC), cyclic olefin polymer (COP), polychlorotrifluoroethylene (PCTFE) and multilaminate materials that can, e.g., provide a hydrophobic surface for the flow channel, the plurality of fluidic harbors, or a combination thereof. In some embodiments, a portion of a device of the present disclosure (e.g., the body) can include COP. In some embodiments, a portion of a device of the present disclosure (e.g., the body) can include COC. In some embodiments, a portion of a device of the present disclosure (e.g., the body) can include COC and COP.

The surface properties of the devices (e.g., channels and/or fluidic harbors) can be tailored for a specific application. For example, some or all surfaces of the devices can be hydrophobic or hydrophilic. In some embodiments, certain surfaces can be hydrophobic and certain surfaces can be hydrophilic. In certain embodiments, some or all of the surfaces can be fluorophilic. The surfaces that are hydrophilic or hydrophobic can be designed so as to allow loading of oils in certain channels and/or fluidic harbors and aqueous solution in certain channels and/or fluidic harbors in the device.

In some embodiments, some or all of the fluidic harbors and/or channels can be modified with chemical or biological reagents to render the surfaces in contact with fluids preferential for wetting by a selected fluid (e.g., either the first or third fluid described above, such as a polybutene). For example, the surfaces can be modified to allow a polybutene (e.g., the first fluid) to preferentially wet the channel and the fluidic harbors. This wetting can prime the surface of the device, which can, for example, facilitate discretization because this makes it easier for the first fluid (e.g., a polybutene) to be drained and displaced by the second fluid (e.g., an aqueous solution) and more difficult for the third fluid (e.g., a polybutene) to displace the second fluid.

### Fluids for Self-Digitization of Sample Volumes

A diverse variety of fluids or liquids can be used with the various devices, systems and methods of the present disclosure. The various fluids, e.g., can include water-based or aqueous solutions. In some embodiments, the fluids can include liquids that are sparingly soluble in aqueous solutions. For example, the fluids can include oils, such as polybutene, fluorinated oils, hydrocarbon oils, silicone oils, or mineral oils. Organic solvents can also be used. In some aspects, devices of the present disclosure can function as at least a two-phase system, utilizing two or more immiscible fluids. For example, a first fluid, e.g., an oil phase fluid, such as polybutene, can initially fill a device to displace any air. The first fluid can be selected to preferentially wet the device surface relative to a second fluid. The second fluid, which is typically immiscible with the first fluid, e.g., an aqueous phase containing the sample of interest, can then flow through the device and enter the fluidic harbors, displacing the oil phase. A third fluid, which can but need not be the same as the first fluid, and which can but need not be miscible with the first fluid, and which is typically immiscible with the second fluid can then be flowed through the device to displace the aqueous phase within the main channels but not the fluidic harbors. In certain aspects, the fluidic harbors can serve as shelters to isolate and digitize individual fluidic packets of the aqueous phase within the fluidic harbors. The fluidic harbors can, though not necessarily required, be substantially occupied by the aqueous phase such that the fluidic packets assume substantially the shape of the fluidic harbor and the volume of the fluidic packet can, essentially, be defined by the dimensions of the harbor. For example, if a fluidic harbor is rectangular shaped, the fluidic packet contained within, should substantially assume a rectangular shape.

In one embodiment, the oil phase used as the first and/or third fluid is polybutene based. In another embodiment, the oil phase is mineral oil based. In another embodiment, the oil phase is fluorocarbon based. In another embodiment, the oil phase is a silicone oil based. Other embodiments can use other “oil” phases or alternative materials. The first/third fluid also typically includes a surfactant and/or wetting agent to improve desired interaction with the device surface and with the second fluid. In certain embodiments, the first and the third fluid are identical. In other embodiments, the first and third fluid can be composed of the same base material, but have different surfactant/additive concentrations or compositions. When a plurality of oil phases are used in a given method of operation (e.g., the first fluid, the third fluid and/or the fourth fluid), the compositions of the oil phases can be the same or different. Each of the oil phase compositions is independently selected regardless of the composition of the other oil phases in use. While in still other embodiments, the third fluid can be of a completely different composition and can but need not be miscible with the first fluid. In certain embodiments, the first and/or third fluid can contain components that interact with the second fluid and/or components within the second fluid.

In some aspects, a fourth fluid is provided. In further aspects, the fourth fluid comprises an oil, such as a fluorinated oil, a hydrocarbon oil, a silicone oil, or a combination thereof. In certain aspects, the fourth fluid comprises a polybutene. In still further aspects, a fluid that is compatible with an amplification reaction, such as PCR or isothermal amplification, is used as the fourth fluid. In some aspects, the fourth fluid can be used to displace the third fluid in the flow channel before beginning an amplification reaction to amplify a digitized analyte. The fourth fluid may be miscible or immiscible with any of the first, second and/or the third fluids. In certain aspects, the fourth fluid may be the same as the first fluid.

In some embodiments, a fifth fluid is provided. In some aspects, the fifth fluid comprises an oil, such as a fluorinated oil, a hydrocarbon oil, a silicone oil, or a combination thereof. In certain aspects, the fifth fluid comprises a polybutene. In certain aspects, the fifth fluid can be used to flush the first, second, third and/or fourth fluid from the device.

In some aspects, any of the first, second, third, fourth and fifth fluids can be provided to a fluid inlet port on the microfluidic device. The first, second, third, fourth and fifth fluids can be provided to the same fluid inlet port or to different fluid inlet ports. In certain aspects, the first, second, third, fourth and fifth fluids are provided to a device in a sequential order that is different from the sequential orders provided herein. The first, second, third, fourth and fifth fluids can be provided to a device in any order, and any of the first, second, third, fourth and fifth fluids may be omitted in some aspects of the method. The sequential orders described herein are exemplary and non-limiting to the practice of the disclosed methods.

In some aspects, the second fluid comprises an aqueous solution. In certain aspects, the methods further comprise providing the first fluid to the fluid inlet port or to a different fluid inlet port on the microfluidic device. In other aspects, the methods further comprise providing a third fluid to the fluid inlet port or to a different fluid inlet port on the microfluidic device, wherein the third fluid comprises an oil phase. In further aspects, the third fluid comprises a fluorinated oil, a hydrocarbon oil, a silicone oil, or a combination thereof. In certain aspects, the third fluid comprises a polybutene.

In various aspects, the present disclosure provides methods for introducing a fluid into a microfluidic device, the method comprising: obtaining a microfluidic device according to the present disclosure; and introducing a first fluid into the flow channel of the microfluidic device.

The terms “providing” and “introducing” are used interchangeably herein to refer to the movement of fluid into or through a structure, such as for example an inlet or a channel.

In some aspects, the first fluid comprises an oil. In further aspects, the oil is selected from a polybutene, a fluorinated oil, a hydrocarbon oil, a silicone oil, or a combination thereof.

In various aspects, the present disclosure provides methods for introducing a fluid into a microfluidic device, the method comprising: obtaining a microfluidic device according to the present disclosure; and introducing a second fluid into the flow channel of the microfluidic device, wherein the second fluid is an aqueous solution.

In some aspects, the second fluid comprises an analyte and the method further comprises performing an analysis of the analyte within at least one of the fluidic harbors. In certain aspects, the analyte comprises a biological material. In further aspects, the biological material is selected from a cell, a bacteria, a virus, a prion, a nucleic acid, a protein, an expressed product of a genetic material, a crystallizing molecule, a particle, or a combination thereof. In yet further aspects, the second fluid comprises a first nucleic acid molecule and a second nucleic acid molecule and the method further comprises distributing the first nucleic acid molecule into a first fluidic harbor, wherein the first fluidic harbor does not comprise the second nucleic acid molecule. In some embodiments, the biological material comprises a nucleic acid conjugated to a protein. In some embodiments, the biological material comprises a nucleic acid conjugated to an antibody.

In some aspects, the present methods further comprise introducing a first fluid into the flow channel of the microfluidic device, wherein the first fluid is introduced into the flow channel before the second fluid is introduced into the flow channel. In some aspects, the first fluid comprises an oil. In further aspects, the oil is selected from a polybutene, a mineral oil, a fluorinated oil, a hydrocarbon oil, a silicone oil, or a combination thereof.

In some aspects, the methods further comprise introducing a third fluid into the flow channel of the microfluidic device. In certain aspects, the third fluid is introduced into the flow channel after the second fluid is introduced into the flow channel. In other aspects, the first fluid is an oil, the second fluid is an aqueous solution, and the third fluid is an oil. In certain aspects, the first fluid comprises a polybutene, the second fluid is an aqueous solution, and the third fluid comprises a polybutene.

In some aspects, the methods further comprise introducing a fourth fluid into the flow channel of the microfluidic device. In certain aspects, the fourth fluid is introduced into the flow channel after the first fluid is introduced into the flow channel and before the second fluid is introduced into the flow channel. In other aspects, the fourth fluid is introduced into the flow channel after the second fluid is introduced into the flow channel and before the third fluid is introduced into the flow channel. In some aspects, the fourth fluid is introduced into the flow channel after the third fluid is introduced into the flow channel. In further aspects, the first fluid is an oil, the second fluid is an aqueous solution, the third fluid is an oil, the fourth fluid is an oil, and the fifth fluid is an oil, and wherein the first, third, fourth, and fifth fluids are independently the same or different from one another. In yet further aspects, each of the oils are independently selected from a polybutene, a mineral oil, a fluorinated oil, a hydrocarbon oil, a silicone oil, or a combination thereof.

In some aspects, the methods further comprise introducing a fifth fluid into the flow channel of the microfluidic device. In certain aspects, the fifth fluid is introduced into the flow channel after the second fluid is introduced into the flow channel. In other aspects, the fifth fluid is introduced into the flow channel after the third fluid is introduced into the flow channel. In some aspects, the method further comprises introducing a fourth fluid into the flow channel, wherein the fourth fluid is introduced into the flow channel after the first fluid is introduced into the flow channel and before the second fluid is introduced into the flow channel. In certain aspects, the first fluid is an oil, the second fluid is an aqueous solution, the third fluid is an oil, the fourth fluid is an oil, and the fifth fluid is an oil, and wherein the first, third, fourth, and fifth fluids are independently the same or different from one another. In further aspects, each of the oils are independently selected from a polybutene, a mineral oil, a fluorinated oil, a hydrocarbon oil, a silicone oil, or a combination thereof.

In certain embodiments, the fluids (e.g., aqueous solutions) herein can contain a variety of analytes that include but are not limited to chemicals, biochemicals, genetic materials (e.g., DNA, RNA, etc.), expressed products of genetic materials (e.g., proteins and/or metabolites), crystallizing molecules, biological cells, exosomes, mitochondria, drugs, biological particles that circulate in peripheral blood or lymphatic systems, rare cells, or particles. Possible aqueous samples that can be used, e.g., as the second fluid, include but are not limited to various PCR and RT-PCR solutions, isothermal amplification solutions such as for LAMP, RT-LAMP, or NASBA, blood samples, plasma samples, serum samples, solutions that contain cell lysates or secretions or bacterial lysates or secretions, and other biological samples containing proteins, bacteria, nucleic acids, nucleic acids conjugated to proteins, nucleic acids conjugated to antibodies, viral particles and/or cells (eukaryotic, prokaryotic, or particles thereof) among others. In certain embodiments, the aqueous solutions can also contain surfactants or other agents to facilitate desired interactions and/or compatibility with immiscible fluids (e.g., the first/third fluid) and/or the material of the device. In certain embodiments, the aqueous solutions loaded on the devices can have cells expressing a malignant phenotype, fetal cells, circulating endothelial cells, tumor cells, cells infected with a virus, cells transfected with a gene of interest, or T-cells or B-cells present in the peripheral blood of subjects afflicted with autoimmune or autoreactive disorders, or other subtypes of immune cells, or rare cells or biological particles (e.g., exosomes, mitochondria) that circulate in peripheral blood or in the lymphatic system or spinal fluids or other body fluids. The cells or biological particles can, in some circumstances, be rare in a sample and the discretization can be used, e.g., to spatially isolate the cells thereby allowing for detection of the rare cells or biological particles. In certain embodiments, the aqueous solutions can also contain detectable agents. In some embodiments, the detectable agents are fluorescent or luminescent agents. In certain embodiments, the detectable agents are capable of labeling a nucleic acid sequence.

In some aspects, an apparatus comprising one or more of the devices disclosed herein is provided.

### Methods for Self-Digitization of Sample Volumes

In certain embodiments, the present disclosure includes sequences for loading the fluidic harbors. The device operation can include providing a first fluid in the reservoir, channel and fluidic harbors. In certain embodiments, the first fluid can include an oil phase that can be introduced to device, e.g., to displace any air bubbles. In certain embodiments, the first fluid can comprise a polybutene. In certain embodiments, a second fluid such as an aqueous sample is provided through the inlet/inlet reservoir, via some loading mechanism, and distributed by the branching inlet to the main channels where it then enters the fluidic harbors, displacing the first fluid (e.g., the oil phase). In certain embodiments, drainage channels facilitate oil phase drainage. After the aqueous sample is loaded, a third fluid, which can be the same oil phase as the first fluid or a different oil phase, is provided that displaces the aqueous sample from the channels, but not the fluidic harbors. In some embodiments, the first fluid comprises polybutene; in other embodiments, the third fluid comprises polybutene; in some embodiments, the first fluid and the third fluid comprise polybutene. This isolates or compartmentalizes the aqueous sample into discrete volumes determined by the fluidic harbor dimensions. Such isolation or compartmentalization can be characterized as digitization or discretization. The compartmentalized volumes can be referred to as discretized or digitized volumes. In one embodiment, all aqueous sample is compartmentalized into fluidic harbors resulting in “loss-less” filling, though not all fluidic harbors would be fully filled. In another embodiment all fluidic harbors are fully filled, but some sample is lost into the outlet/outlet reservoir.

Drainage channels can impact the rate of oil phase displacement by the aqueous sample and can impact the device loading rate as well as the completeness of filling of the fluidic harbors. In some embodiments, drainage channels are utilized in side-harbor designs. While side-harbor and bottom-harbor designs can share many similar features, the relative size of the connection between the fluidic harbor and the main channel can affect the rate of oil phase drainage/fluid exchange. Bottom-harbor designs may not benefit from drainage channels, while side-harbor designs can benefit from their presence.

Digitization can be achieved using a wide range of channel loading or filling mechanisms. Any suitable channel filling mechanism or channel filling component can be used to move a fluid through the flow channel. Different loading mechanisms include, but are not limited to, syringe pump driven flow, positive pressure or negative (vacuum) pressure driven flow, and by centrifugal force driven flow. In some embodiments, fluid can be moved through the channels by using pressure filling. In certain embodiments, fluid can be moved through the channels by using a positive pressure. As a non-limiting example, fluid can be moved through the channels by applying a positive pressure to an inlet port of a device. In some embodiments, fluid can be moved through the channels by using a negative pressure. As a non-limiting example, fluid can be moved through the channels by applying a negative pressure to an outlet port of a device. In some embodiments, fluid can be moved through channels by using a vacuum. In specific embodiments, fluid can be moved through the channels by providing a vacuum to the outlet port of a device. In some aspects, loading can be carried out by rotating a microfluidic device herein about its central axis so as to drive fluid loading into channels through centrifugal force. Fluid pressure can also be applied, using e.g., positive or negative pressure, to drive fluid into fluid inlet ports on the device. In other aspects, the channel filling component can be a source of air pressure, pneumatic pressure, hydrodynamic or hydraulic pressure, or the like, or a combination thereof. In further aspects, the source of pressure is in fluidic communication with a fluid inlet port or a fluid outlet port of the device.

As discussed above and herein, rotation of the microfluidic device can be performed in various ways. In various embodiments, rotating the microfluidic device comprises: providing a rotor assembly comprising a central axis and a plurality of receptacles arranged radially around the central axis; positioning the microfluidic device in one of the plurality of receptacles such that the proximal body portion of the microfluidic device is positioned near the central axis and the distal body portion of the microfluidic device is positioned away from the central axis; and rotating the rotor assembly around the central axis, thereby rotating the microfluidic device.

### Systems for Self-Digitization of Sample Volumes

The devices and methods described herein can further include detection, imaging and/or analysis. In some aspects, the present disclosure can include analytical systems that can include, e.g., a chamber configured to accept or house the microfluidic devices described herein. In some aspects, the system is configured for housing, rotating, and processing a plurality of microfluidic devices. The analytical systems can also include a plurality of fluid reservoirs for containing one or more fluids. The plurality of fluid reservoirs can be, e.g., configured to provide the fluid to the fluid inlet port of the microfluidic devices. The systems can also include components to facilitate loading of fluids onto the devices. For example, the systems can include a fluid pressuring unit configured to apply pressure to the flow channel so as to drive or urge the fluid through the flow channels. The systems can also include a rotation component (e.g., a motor) that couples to the devices and is configured to spin or rotate the devices, e.g., about a central axis of the microfluidic device. In some aspects the rotation component is part of a modified optical disc drive, such as a compact disc (CD) drive, a digital video disc (DVD) drive, a Blu-ray drive, or a modified version thereof, or a combination thereof.

A variety of reactions can be carried out using the analytical systems. For reactions, the systems can include a heat-control component configured to apply heat to at least one of the fluidic harbors. Detection and/or imaging can be provided using an optical detection component for optically analyzing at least one of the fluidic harbors of the microfluidic device. The systems can also be coupled to a computer system that, e.g., can include a processing unit configured to control the heat control component and the optical detection component, and being configured to store data generated from the optical detection component.

In some embodiments, a basic wide area microscopic imaging setup can be used to acquire bright field and/or fluorescent images of SD devices. For devices that have multiple arrays, an automated stage/positioning system can be used to quickly image all arrays in a single device, such as centrifugal devices with multiple arrays, or devices based off of 96-well plates or other automation supporting layouts. In another embodiment the imaging can be coupled to the rotational position of a device using rotary encoders that have high rotational resolution, which would enable imaging of devices while they are spinning. This can further speed data acquisition and can also be used to monitor loading of centrifugal devices in real time.

In various aspects, the present disclosure provides analytical systems comprising: a chamber configured to accept a microfluidic device according to the present disclosure; a fluid introducing component configured to introduce a fluid through at least a portion of a flow channel; an optical detection component configured to optically analyze a fluidic harbor; and a processing unit configured to control the optical detection component, and configured to store data generated from the optical detection component.

In some aspects, the fluid introducing component is configured to move a fluid through at least one of a plurality of flow channels. In other aspects, the fluid introducing component is selected from an air pressure source, a pneumatic pressure source, a hydraulic pressure source, or a combination thereof, and wherein the fluid introducing component comprises a source of positive or negative pressure sufficient to move a fluid into a flow channel. In some embodiments, fluid can be moved into the flow channel by using pressure filling. In certain embodiments, fluid can be moved into the flow channel by using a positive pressure. As a non-limiting example, fluid can be moved into the flow channel by applying a positive pressure to an inlet port of a device. In some embodiments, fluid can be moved into the flow channel by using a negative pressure. As a non-limiting example, fluid can be moved into the flow channel by applying a negative pressure to an outlet port of a device. In some embodiments, fluid can be moved into the flow channel by using a vacuum. In specific embodiments, fluid can be moved into the flow channel by providing a vacuum to the outlet port of a device. In further aspects, the fluid introducing component is configured to move the fluid at least partially through a flow channel by capillary action, wicking, centrifugal force or a combination thereof. In further aspects, the optical detection component is configured to optically analyze a plurality of fluidic harbors. In some aspects, the present apparatuses further comprise a heat-control component configured to apply heat to at least one of the fluidic harbors. In some aspects, the optical detection component comprises an imaging device, an optical disc drive, a laser scanner, or a combination thereof.

Various embodiments of the present disclosure provide systems for self-digitization, processing, and/or analysis of fluidic samples. In some embodiments, a system is configured to discretize a fluidic sample by driving fluid into the fluidic harbors of a microfluidic device by centrifugation. In certain embodiments, the system configured for discretizing a plurality of fluidic samples includes a rotor assembly configured to receive one or more microfluidic devices, and a rotary actuator configured to rotate the rotor assembly about an axis of rotation (e.g., a central axis of the rotor assembly) in order to generate centrifugal forces for driving fluid into the fluidic harbors of the microfluidic device(s). The systems herein advantageously provide a simple and convenient format for simultaneous discretization of multiple fluidic samples suitable for high-throughput processing and analysis.

A rotor assembly for self-digitization of fluidic samples can be configured in a variety of ways. In some embodiments, a rotor assembly includes a central axis and a plurality of receptacles arranged radially around the central axis. The rotor assembly can include any number of receptacles, such as 2, 3, 4, 5, 6, 7, 8, 9, 10, 11, 12, or more receptacles. The distance between the proximal end of each of the plurality of receptacles and the central axis is within a range from about 10 mm to about 500 mm, from about 20 mm to about 300 mm, or from about 30 mm to about 200 mm, in some embodiments. The receptacles are each shaped to receive one or more microfluidic devices of the present disclosure. For instance, in various embodiments, a receptacle is sized and/or shaped to substantially match the size and/or shape of a device body of a microfluidic device (e.g., has a length of approximately 127.8 mm and a width of approximately 85.5 mm, or a length of approximately 100 mm and a width of approximately 75 mm). In certain embodiments, the receptacles are shaped to removably receive and couple to the microfluidic devices, e.g., via interference fits, snap fits, fasteners, latches, clamps, or other suitable coupling mechanisms.

In some embodiments, the receptacles are arranged such that the proximal body portion of a received microfluidic device is positioned near the central axis of the rotor assembly, while the distal body portion of the device is positioned away from the central axis of the rotor assembly. Accordingly, in such embodiments, rotation of the rotor assembly causes fluid to be driven through the device from the proximal portion to the distal portion. As discussed above and herein, the microfluidic device optionally includes tapering flow channels with a decreasing cross-sectional dimension from the proximal portion to the distal portion in order to promote uniform fluid flow during centrifugation.

The rotary actuator can be any actuation mechanism suitable for rotating the rotor assembly about an axis of rotation, such as a brushless direct current motor, a brushed direct current motor, a servo motor, or a stepper motor. In certain embodiments, the rotary actuator is configured to rotate the rotor assembly at approximately 10 RPM, 50 RPM, 100 RPM, 200 RPM, 300 RPM, 400 RPM, 500 RPM, 600 RPM, 700 RPM, 800 RPM, 900 RPM, 1000 RPM, 1500 RPM, 2000 RPM, 2500 RPM, 3000 RPM, 3500 RPM, 4000 RPM, 4500 RPM, or 5000 RPM. In various embodiments, the rotational speed of the rotary actuator is configured for driving fluid through the microfluidic devices in order to discretize fluidic samples. Optionally, the rotational speed is configured for other applications, such as the imaging procedures discussed further herein. In certain embodiments, the rotation of the rotary actuator is controlled by one or more processors configured with instructions for controlling the operation of the self-digitization system, as discussed further herein.

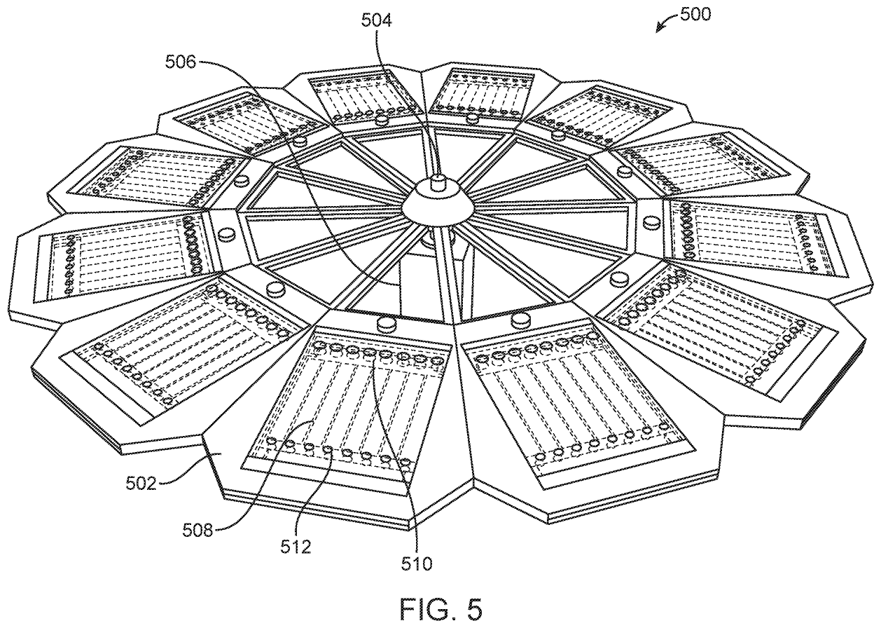

FIG. 5 illustrates a rotor assembly 500 of a system for self-digitization of fluidic samples, in accordance with embodiments. The rotor assembly 500 includes a plurality of receptacles 502 (e.g., 12 receptacles) arranged radially about a central axis 504. The central axis 504 is coupled to a rotary actuator 506, depicted herein as being positioned underneath the rotor assembly 500. Each receptacle 502 is shaped to receive a corresponding microfluidic device 508. The microfluidic devices 508 can be similar to any embodiment of the devices discussed herein, such as the device 100. In certain embodiments, the devices 508 are positioned in their corresponding receptacles 508 such that the proximal portion 510 of the device 508 is oriented towards the central axis 504 and the distal portion 512 of the device 508 is oriented away from the central axis 504. As discussed above and herein, in some embodiments, the fluid inlet ports and fluid outlet ports of the device 508 are located at the proximal portion 510, the outlet reservoir is located at the distal portion 512, and the flow channels extend from the proximal portion 510 to the distal portion 512.

In some embodiments, a rotor assembly is integrally formed as a single piece such that the receptacles cannot be decoupled from each other without damaging the assembly. In such embodiments, the number of receptacles is fixed. In alternative embodiments, the rotor assembly includes removably coupled receptacles such that the number of receptacles can be adjusted according to user preference. Removably coupled receptacles can be attached to the rotor assembly in a variety of ways, including but not limited to fasteners (e.g., screws, pins), interlocking elements, snap fits, interference fits, latches, clamps, and the like.

Various embodiments of the present disclosure provide systems and devices configured to perform other functionalities in addition to self-digitization of fluidic samples. In some embodiments, a system for self-digitization as discussed herein also incorporates components configured to perform one or more of the following functions: heating of one or more fluidic samples in a microfluidic device, cooling of one or more fluidic samples in a microfluidic device, measuring a property of one or more fluidic samples in a microfluidic device, and/or imaging one or more fluidic samples in a microfluidic device. For instance, various embodiments of the systems herein are configured to perform discretization, thermal cycling, and imaging of a plurality of fluidic samples, e.g., for dPCR applications. Such multifunctional systems provide a convenient and compact unified platform for high-throughput discretization, processing, and analysis of fluidic samples.

The various components (e.g., rotary actuator, heating device, cooling device, temperature sensor, ventilation assembly, imaging device, illumination source, etc.) of the self-digitization systems described herein can be configured in multiple ways. In some embodiments, one or more of the components herein are positioned above the rotor assembly and received microfluidic devices. In some embodiments, one or more of the components herein are positioned below the rotor assembly and received microfluidic devices. In some embodiments, one or more of the components herein are positioned on the rotor assembly, such as coupled to the rotor assembly.

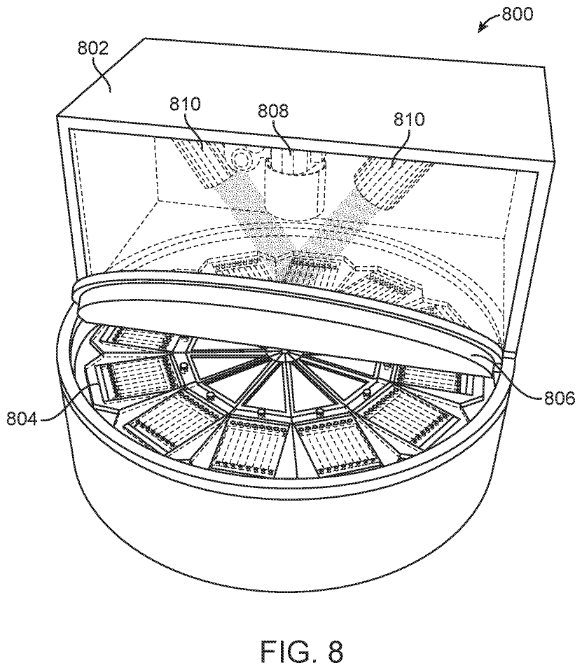

FIG. 8 illustrates an exemplary multifunctional system 800 for sample discretization, heating, and imaging, in accordance with embodiments. The system 800 can be used in combination with any embodiment of the methods and devices herein. The system 800 includes a housing 802 that partially or wholly encloses a rotor assembly 804 for receiving a plurality of microfluidic devices. In some embodiments, the housing 802 includes a lid 806 allowing access to the rotor assembly 804 (e.g., for loading and unloading microfluidic devices). The rotor assembly 804 is coupled to a rotary actuator (not shown) for rotating the rotor assembly 804, e.g., in order to drive fluid into the fluidic compartments of the received microfluidic devices to discretize a sample. A plurality of heating devices (not shown) are positioned below the rotor assembly 804 for heating the microfluidic devices, e.g., in accordance with a thermal cycling procedure. In some embodiments, the number of heating devices corresponds to the number of receptacles in the rotor assembly 804, such that each heating device is used to apply heat to a single respective microfluidic device. The system 800 also includes an imaging device 808 for imaging the microfluidic devices in the rotor assembly 804 and one or more illumination sources 810 for illuminating the microfluidic devices during imaging. In some embodiments, the imaging device 808 and illumination sources 810 are positioned above the rotor assembly 804.

In some aspects, the systems further comprise a plurality of fluid reservoirs for containing a fluid wherein the plurality of fluid reservoirs are configured to provide the fluid to the fluid inlet port of the microfluidic device. In some aspects, the systems further comprise a second fluid reservoir and a third fluid reservoir, wherein the second fluid reservoir comprises a second fluid and the third fluid reservoir comprises a third fluid, wherein the second fluid comprises an aqueous solution and the third fluid comprises an oil phase. In certain aspects, the third fluid comprises a polybutene. In certain aspects, the microfluidic device is loaded with a first fluid and the second fluid, wherein the first fluid comprises an oil phase. In some aspects, the oil phase comprises a polybutene. In further aspects, the optical detection component is configured to detect an analyte present in a fluidic harbor. In other aspects, the optical detection component and the processing unit are configured to determine a volume of the aqueous solution in at least some of the fluidic harbors. In some aspects, each of the fluidic harbors are in fluidic communication with a flow channel through an opening conduit. In certain aspects, at least one of the fluidic harbors comprises at least one channel in fluidic communication with the flow channel.

### Methods of Performing a Digital Assays Using Droplet Systems

In various aspects, the present disclosure provides methods for performing a digital assay. A plurality of compartmentalized volumes may be produced. In certain embodiments, the compartmentalized volumes comprise polydisperse droplets. In specific embodiments, the compartmentalized volumes comprise a plurality of polydisperse droplets. In some embodiments, the compartmentalized volumes comprise fluidic harbors as disclosed herein. In certain embodiments, the compartmentalized volumes comprise wells as disclosed herein. At least some of the compartmentalized volumes may comprise a sample. The sample may be amplified. The sample may be labeled with a detectable agent. The volume of the compartmentalized volume may be determined. The presence or absence of the detectable agent in the compartmentalized volume may be determined. The concentration of the sample in the compartmentalized volumes may be determined based on the presence or absence of the detectable agent in a plurality compartmentalized volumes.

In some embodiments, performing a digital assay comprises ELISA amplification. In some embodiments, performing a digital assay comprises NASBA amplification. In some embodiments, performing a digital assay comprises LAMP amplification. In some embodiments, performing a digital assay comprises digital PCR amplification.

In certain embodiments, the compartmentalized volume is a droplet. In specific embodiments, the compartmentalized volume comprises a plurality of polydisperse droplets. In some embodiments, the compartmentalized volume is a fluidic harbor as disclosed herein.

The term “compartmentalized volume” refers to the volume of a substance that is substantially encompassed by a surrounding element. In certain embodiments, the term “sample volume” can refer to the compartmentalized volume. Non-limiting examples of a compartmentalized volume include a well or chamber in a microfluidic chip, a droplet (e.g., a water droplet formed in an emulsion, on the surface of a chip, or in a microfluidic device), or a fluidic harbor (e.g., an aqueous volume formed by self-digitization as described herein).

### Materials and Methods for Formation of Droplets

The plurality of polydisperse droplets may comprise a first fluid and a second fluid. The first fluid may be immiscible in the second fluid. An emulsion of polydisperse droplets may be formed by agitating a solution comprising a first fluid and a second fluid, wherein the first fluid is immiscible in the second fluid. To form the emulsion of polydisperse droplets, a solution comprising a first fluid and a second fluid may be agitated. The first fluid may be immiscible in the second fluid. The emulsion may be agitated in a third fluid. The third fluid may be immiscible in the second fluid, thereby forming a double emulsion. The fluid(s) may be agitated in many ways such as shaking, vortexing, sonicating, mixing with magnets, extrusion, flow focusing or a combination thereof. The agitation may be sufficient to form an emulsion. Extrusion, for example, may comprise pipetting the fluid, wherein the pipetting is sufficient to produce the emulsion. The agitating may occur in a microfluidic device. The agitation may be, for example, vortexing. The emulsion produced may comprise an aqueous phase and a non-aqueous phase. The first fluid may comprise water and the second fluid may comprise an oil phase. In certain embodiments, the oil phase comprises a polybutene. The first fluid may comprise water, the second fluid may comprise an oil phase, and the third fluid may comprise water. In certain embodiments, the oil phase comprises a polybutene.

The plurality of polydisperse droplets may comprise a plurality of emulsions. The plurality of emulsions may be prepared by combining three or more immiscible fluids. The first fluid may be aqueous. The first fluid may comprise sample. The second fluid may comprise an oil phase. The second fluid may comprise a polybutene. The second fluid may comprise an oil phase and the second fluid may be immiscible with the first fluid and the third fluid. In certain embodiments, the oil phase comprises a polybutene. The first fluid may be different from the third fluid. The third fluid may comprise an oil phase and the third fluid may be immiscible with the first fluid and the second fluid. In certain embodiments, the oil phase comprises a polybutene.

The plurality of polydisperse droplets may further comprise a fluid interface modification element. The fluid interface modification element may comprise a surfactant. The fluid interface modification element may be selected from a lipid, phospholipid, glycolipid, protein, peptide, nanoparticle, polymer, precipitant, microparticle, a molecule with a hydrophobic portion and a hydrophilic portion, or a combination thereof.

An emulsion of droplets may be formed by agitating a solution comprising a first fluid and a second fluid in a microfluidic device, wherein the first fluid is immiscible in the second fluid. To form the emulsion of droplets, a solution comprising a first fluid and a second fluid may be agitated. The first fluid may be immiscible in the second fluid. The emulsion may be agitated in a third fluid. The third fluid may be immiscible in the second fluid, thereby forming a double emulsion. The fluid(s) may be agitated in a microfluidic device. The first fluid may comprise water and the second fluid may comprise an oil phase. In certain embodiments, the oil phase comprises a polybutene. The first fluid may comprise water, the second fluid may comprise an oil phase, and the third fluid may comprise water. In certain embodiments, the oil phase comprises a polybutene.

In certain embodiments of the present disclosure, the droplets can be formed using a microfluidic device in a continuous-flow fashion either in the T-channel geometry or in the flow focusing geometry, both of which are well known in the art. In another embodiment, the sample can be digitized using microfluidic channels and immiscible fluid phases. In this embodiment, the sample phase is introduced into the channel, followed by an immiscible phase which forms discrete sample volumes that are defined by the geometric dimensions of the side cavities (D. E. Cohen, T. Schneider, M. Wang, D. T. Chiu Anal. Chem. 82, 5707-5717). In certain embodiments, the immiscible phase comprises an oil phase. In preferred embodiments, the immiscible phase comprises a polybutene.

### Analysis, Methods, Systems, and Materials for Performing a Digital Assay

In various aspects, the present disclosure provides methods for performing a digital assay. A plurality of compartmentalized volumes may be produced. In certain embodiments, the compartmentalized volumes comprise polydisperse droplets. In specific embodiments, the compartmentalized volumes comprise a plurality of polydisperse droplets. In some embodiments, the compartmentalized volumes comprise fluidic harbors as disclosed herein. In some embodiments, the compartmentalized volumes comprise wells as disclosed herein. At least some of the compartmentalized volumes may comprise a sample. The sample may be amplified. The sample may be labeled with a detectable agent. The volume of the compartmentalized volume may be determined. The presence or absence of the detectable agent in the compartmentalized volume may be determined. The concentration of the sample in the compartmentalized volumes may be determined based on the presence or absence of the detectable agent in a plurality compartmentalized volumes.

The detectable agent may be fluorescent or luminescent. The detectable agent may comprise one or more of SYBR® green, Evagreen®, SYTO™-9, SYTO™-82, fluorescein, FITC, FAM™, rhodamine, HEX™, VIC®, JOE™, TET™, TAMRA™, ROX™, TRITC, Texas Red®, GFP, phycoerythrin (PE), a cyanine, a cyanine derivative, Cy™3, Cy™3.5, Cy™5, Cy™5.5, PE-Cy™5, Calcein, a BODIPY®, an Alexa Fluor®, a DyLight® Fluor, an ATTO, a Quasar® dye, a Cal Fluor®, a TYE™, a Qdot, a Cy™ dye, a SYSTO, derivatives thereof, a semiconducting polymer, or a semiconducting polymer dot. The sample may comprise a nucleotide.

To amplify the sample, polymerase chain reaction (PCR), rolling circle amplification (RCA), nucleic acid sequence based amplification (NASBA), loop-mediated amplification (LAMP), Recombinase Polymerase Amplification (RPA), Helicase-Dependent Amplification (HAD, or a combination thereof may be performed. Alternatively or in combination, the sample may be amplified by isothermal amplification of nucleotides or variable temperature amplification of nucleotides.

In various aspects, the present disclosure provides compositions for performing digital assays. A composition may comprise a first fluid, a second fluid, a surfactant, and an amplification reagent. The first fluid and the second fluid may be immiscible in each other and may be capable of forming an emulsion when agitated. In some aspects, the composition may further comprise a sample, such as a nucleotide, and/or a detectable agent capable of labeling the sample. The sample may be labeled with the detectable agent.

In addition to aqueous solutions and non-aqueous fluids, surfactants can also be included to, e.g., improve stability of the droplets and/or to facilitate droplet formation. Suitable surfactants can include, but are not limited to, non-ionic surfactants, ionic surfactants, silicone-based surfactants, fluorinated surfactants or a combination thereof. Non-ionic surfactants can include, for example, sorbitan monostearate (Span 60), octylphenoxyethoxyethanol (Triton X-100), polyoxyethylenesorbitan monooleate (Tween 80) and sorbitan monooleate (Span 80). Silicone-based surfactants can include, for example, ABIL WE 09 surfactant. Other types of surfactants generally well known in the art can similarly be used. In some aspects, the surfactant can be present at a variety of concentrations or ranges of concentrations, such as approximately 0.01%, 0.1%, 0.25%, 0.5%, 1%, 5%, or 10% by weight.

The composition may further comprise a detectable agent capable of binding a nucleic acid sample.

The amplification reagent of the composition may be selected from a polymerase chain reaction (PCR) reagent, rolling circle amplification (RCA) reagent, nucleic acid sequence based amplification (NASBA) reagent, loop-mediated amplification (LAMP) reagent, Recombinase Polymerase Amplification (RPA) reagent, Helicase-Dependent Amplification (HDA) reagent, or a combination thereof. The amplification reagent may comprise a PCR reagent such as a thermostable DNA polymerase, a nucleotide, a primer, probe or a combination thereof.

The composition may further comprise a third fluid. The third fluid may be immiscible in the second fluid. The composition may be capable of forming a double emulsion. The first fluid may be aqueous. The first fluid may comprise the amplification reagent. The second fluid may comprise an oil phase. In certain embodiments, the oil phase comprises a polybutene. The second fluid may be immiscible with the first fluid and the third fluid. The first fluid may be different from the third fluid. The third fluid may comprise an oil phase and may be immiscible with the first fluid and the second fluid. In certain embodiments, the oil phase comprises a polybutene.

The present disclosure can be used for any technique in which digital measurements provide useful information about a sample. As such, the methods, systems and devices provided herein can include a volume containing a detectable agent. In certain aspects, the volume can be a well or chamber in a microfluidic device, a droplet (e.g., a water droplet formed in an emulsion or on the surface of a chip), or a fluidic harbor (e.g. an aqueous compartmentalized volume formed by self-digitization) that contains the detectable agent. It will be generally understood that the detectable agent can include a single detectable molecule or a plurality of detectable molecules. Other types of detectable agents can be used, e.g., beads, quantum dots, nanoparticles, and the like. Furthermore, the detectable agent can, for example, be a molecule of interest present in a sample to be analyzed (e.g., a nucleic acid molecule in blood, serum, saliva or other solutions). Alternatively, a detectable agent can be a molecule that associates with a molecule of interest (e.g., the nucleic acid molecule) in the sample, thereby allowing the molecule to be detected. In some aspects, the methods and systems of the present disclosure can be used for amplification-related techniques (e.g., digital PCR) involving digital measurements. For amplification measurements, a volume (e.g., a fluidic harbor) can include a single DNA molecule, for example, but the volume will also contain necessary components that are generally well known to be used for amplification and detection. In some aspects, the detectable agent is fluorescent and, thus, can be detected by fluorescence-based detection methods known in the art. However, other detection methods (e.g., absorbance, chemiluminescence, turbidity, and/or scattering) can be used to analyze the contents of a volume. A variety of detectable agents suitable for the present disclosure are generally well known in the art and can, for example, be found in The Molecular Probes Handbook, 11^{th} Edition (2010).

In some embodiments, the present disclosure provides methods for using digital measurements to determine a concentration of a sample. The methods can include producing a first plurality of compartmentalized volumes having a first volume distribution, wherein at least one of the compartmentalized volumes of the first plurality contains contents from the sample; analyzing a second plurality of compartmentalized volumes having a second volume distribution to determine volumes of the compartmentalized volumes in the second plurality and a number of compartmentalized volumes in the second plurality that contain a detectable agent, wherein the first volume distribution is the same or different than the second volume distribution; and using volumes of the compartmentalized volumes in the second plurality and the number of compartmentalized volumes in the second plurality that contain the detectable agent to determine the concentration of the sample.

Amplification of analyte (e.g., digital PCR) can be carried out simultaneously in all compartmentalized volumes of different volumes (sizes). In some embodiments, amplification of analyte can be carried out in all droplets of different volumes, after which the droplets can be flowed in a single-file format through a flow cytometer or other similar device where the size of the droplet can be determined and the fluorescence from the droplet can be interrogated. In this example device, the presence of amplification product in each droplet is determined based on fluorescence and the size (volume) of each droplet is determined based on the scattering signal from the droplet. In this way, by noting both the size of each droplet and the presence or absence of amplification product in each droplet of a given size, it is possible to back-calculate the original concentration of the analyte present in the sample after interrogating a sufficient number of droplets of different sizes. Because the droplets are of different sizes, for a given dynamic range, the analysis is much faster than if the droplets are all of a similar size for reasons discussed previously.

As described herein, the compartmentalized volumes can be produced having a variety of volume distributions, which can be analyzed using a variety of different methods. In some embodiments, a sample can contain a molecule or molecules of interest that can be analyzed. Discrete volumes of the sample can be generated for analysis via digital measurements. For example, the methods herein can include producing a plurality of compartmentalized volumes having a volume distribution. In some embodiments, the plurality of compartmentalized volumes can be a plurality of droplets. In some embodiments, the plurality of droplets of the sample can be produced in an emulsion that includes combining immiscible fluids, as further described herein. In one example, a sample can include an aqueous solution that includes a molecule of interest (e.g., a nucleic acid molecule). The sample can be mixed with an oil phase to form droplets of the sample suspended in the oil phase. In certain embodiments, the oil phase comprises polybutene. Depending on the method used, the volumes of the plurality of droplets in the emulsion can be randomly distributed along a continuous volume distribution. Furthermore, the ranges of volumes can be controlled by the method used to form the emulsions. For example, extrusion through channel or capillary openings or apertures of a desirable size, or intensity of vortexing, shaking, and/or sonicating can be controlled to produce a desired volume distribution. Selection ranges of volumes can additionally be controlled by selection of a self-digitization chip having a plurality of fluidic harbors. In certain aspects, the volume of the fluidic harbors is known prior to loading.

As one of ordinary skill in the art will appreciate, it is important to mediate the evaporation of an aqueous phase during thermal cycling or isothermal heating. In some embodiments, a vapor barrier is used to minimize evaporation. A vapor barrier is especially important for small volumes (e.g., those used in dPCR), at least because the surface area is greatly increased. In certain embodiments, an oil phase can be used as a vapor barrier. In some embodiments, a polybutene can be used as a vapor barrier. In certain embodiments, a polybutene can be used as a vapor barrier in dPCR. In certain embodiments, a polybutene can be used as a vapor barrier in dLAMP.

As a person of ordinary skill in the art will appreciate, it is important to have efficient heating in thermal cycling or in the application of isothermal heating. In some embodiments, polybutene can improve efficiency of thermal cycling as well as the efficiency of isothermal heating.

A person of ordinary skill in the art will additionally appreciate the importance of the compatibility of an oil phase with other facets of amplification (e.g., dPCR or dLAMP). Compatibility of an oil phase with the compartmentalized volume substrate can prevent adverse effects of using non-compatible liquids during thermal cycling or isothermal heat application. Adverse effects of using a non-compatible liquid in amplification during thermal cycling or isothermal heat application can include, but are not limited to, fogging of the substrate (which can, in turn, adversely affect optics and analysis) and degradation of the substrate (which can, in turn, adversely affect the PCR reaction or imaging of the volume). In certain embodiments, the oil phase is compatible with the compartmentalized volume substrate. In some embodiments, polybutene is compatible with the compartmentalized volume substrate. In certain embodiments, the total light transmittance of the compartmentalized volume substrate following thermal cycling or isothermal heat application in presence of polybutene is greater than 40%, is greater than 50%, is greater than 60%, is greater than 70%, is greater than 75%, is greater than 80%, is greater than 85%, is greater than 86%, is greater than 87%, is greater than 88%, is greater than 89%, is greater than 90%, is greater than 91%, is greater than 92%, is greater than 93%, is greater than 94%, is greater than 95%, is greater than 96%, is greater than 97%, is greater than 98%, or is greater than 99%. In some embodiments, the total light transmittance is measured using wavelengths of visible light. In certain embodiments, the total light transmittance is measured using wavelengths between 300 nanometers and 800 nanometers. In some embodiments, the total light transmittance of the compartmentalized volume substrate following thermal cycling or isothermal heat application in presence of polybutene is compared to the total light transmittance of the compartmentalized volume substrate prior to the corresponding thermal cycling or isothermal heat application, and the change in the total light transmittance is less than 30%, is less than 25%, is less than 20%, is less than 15%, is less than 10%, is less than 9%, is less than 8%, is less than 7%, is less than 6%, is less than 5%, is less than 4%, is less than 3%, is less than 2%, is less than 1%, or is less than 0.5%. In certain embodiments, there is no measurable change in total light transmittance of the compartmentalized volume substrate when comparing it prior to and following thermal cycling or isothermal heat application in presence of polybutene.

As one of ordinary skill in the art will appreciate, it is important that an oil phase is compatible with a detectable agent. As a non-limiting example, an oil phase can be compatible with a detectable agent if it does not significantly interfere with the fluorescence or optical properties of the detectable agent. In certain embodiments, the oil phase is compatible with the detectable agent. In some embodiments, polybutene is compatible with the detectable agent. In some embodiments, polybutene is compartible with nucleic acid amplification. In certain embodiments, polybutene is compatible with digital nucleic acid amplification, such as digital PCR and digital LAMP. As used herein, compatibility with nucleic acid amplification indicates that polybutene does not inhibit amplification in a way that would negatively affect quantification or determination of the concentration of the analyte. In certain embodiments, compatibility of amplification with polybutene comprises an ability to detect the amplified nucleic acid product. In some embodiments, the amplifying of the nucleic acid is compatible with polybutene, wherein being compatible with polybutene comprises an ability to detect the amplified nucleic acid product. In certain embodiments, being compatible with polybutene further comprises an analytical result having less than 20% variance from the true value. In some embodiments, the amplifying of the nucleic acid is compatible with polybutene wherein being compatible with polybutene comprises an analytical result having less than 50% variance from the true value. In certain embodiments, the variance is less than 45% from the true value, less than 40% from the true value, less than 35% from the true value, less than 30% from the true value, less than 25% from the true value, less than 20% from the true value, less than 15% from the true value, less than 10% from the true value, less than 9% from the true value, less than 8% from the true value, less than 7% from the true value, less than 6% from the true value, less than 5% from the true value, less than 4% from the true value, less than 3% from the true value, less than 2% from the true value, or less than 1% from the true value. In certain embodiments, compatibility with polybutene further comprises an ability to detect the detectable agent. As provided herein, the term “variance” of percentage is intended to describe a parameter for values that are greater than or less than a true value. For example, if the true value for a concentration is 0.2 mM, a variance of less than 20% would include values from greater than 0.16 mM to less than 0.24 mM. A person of skill will appreciate that as used herein, variance provides a parameter for measuring accuracy having greater than or less than a stated percentage (e.g., a value with a variance of less than 20% provides for the true value±less than 20%). In some embodiments, the analytical result comprises visualizing the amplified product. In certain embodiments, the analytical result comprises determining the presence or absence of the amplified product. In some embodiments, the analytical result comprises determining the relative size of the amplified product. In certain embodiments, the analytical result comprises determining the size of the amplified product. In some embodiments, the analytical result comprises determining the concentration of a sample.

As will be appreciated by one of ordinary skill in the art, the ranges for and volumes within a volume distribution will depend on a variety of factors for a given analysis. In some embodiments, the volume distributions of the compartmentalized volumes can include a volume range from about 100 nanoliters (nL) to about 1 femtoliter (fL), from about 10 nL to about 10 fL, from about 1 nL to about 100 fL, from about 100 nL to about 1 picoliter (pL), from about 10 nL to about 10 pL, from about 1 nL to about 1 pL. Depending on the selected factors for producing compartmentalized volumes, it is routine to define the upper and lower boundaries of a volume distribution. There can be ranges of volumes in the volume distributions. For example, volumes in the distributions can range by more than a factor of 2, by more than a factor of 10, or by more than a factor of 100 and by other factors. By ranging by a factor of 2, the lower boundary of the volume distribution can be, e.g., 10 nL with an upper boundary of 20 nL. Similarly, by ranging by a factor 10, the lower boundary of the volume distribution can be, e.g., 10 nL with an upper boundary of 100 nL.

In addition to producing a first plurality of compartmentalized volumes having a first volume distribution, the present disclosure further includes analyzing a second plurality of compartmentalized volumes having a second volume distribution. Analyzing the second plurality of compartmentalized volumes can include, e.g., determining volumes of the compartmentalized volumes in the second plurality. This volume determination can be done using a variety of methods (e.g., using scattering and/or microscopy). In some embodiments, individual volumes of all of the compartmentalized volumes in the plurality may be determined. In some embodiments, only individual volumes of some of the compartmentalized volumes may be determined. Analyzing the compartmentalized volumes can also include determining the number of compartmentalized volumes that include a detectable agent (i.e., one or more detectable agents) further described herein. It is further noted that the second plurality of compartmentalized volumes is based on the same compartmentalized volumes produced as the first plurality of compartmentalized volumes. Thus, the first volume distribution can be the same or different (e.g., narrower) than the second volume distribution. If the distributions are the same, then each compartmentalized volume in the first plurality will be included in the second plurality. In certain embodiments, the second volume distribution is narrower than the first volume distribution. For example, compartmentalized volumes can be produced having a volume distribution ranging from about 1 fL to about 100 nL. Depending, for example, on the concentration of the sample, analysis for digital measurements may be conducted for a volume distribution ranging from 1 fL to about 1 nL, in which the second volume distribution is narrower than the first volume distribution.

In certain aspects, the compartmentalized volumes of the present disclosure have a continuous volume distribution. As provided herein, the term “continuous volume distribution” is intended to describe a distribution of volumes that vary continuously, rather than by pre-defined discrete steps, across the volume distribution. For example, chip-based platforms can include well or droplet volumes that cover a volume distribution defined by pre-defined, discrete steps fabricated as part of the chip. That is, a chip can be made to have volumes present at 100 nL, 10 nL, and 1 nL, with no other volumes present in between those discrete steps. In contrast, a continuous volume distribution in not pre-defined (i.e., the volume distribution is undefined prior to producing or forming compartmentalized volumes). The continuous volume distributions can, for example, be produced via emulsification, as described further herein. In certain embodiments, the continuous volume distributions can be produced via emulsification by extrusion through a channel opening or aperture. In emulsions, the volumes (e.g., droplet volumes) have a discrete volume but the exact droplet volume of a particular droplet in the distribution is undefined prior to producing the droplets (i.e., not pre-defined by fabrication techniques) and the volumes are randomly distributed along the continuous volume distribution. According to the present disclosure, an emulsification system can be produced by physical agitation, such as for example vortexing or shaking the sample, or extrusion through a channel opening or aperture. An upper and lower boundary for droplet volumes can be modified by the forces imparted on the emulsion (e.g., by the speed of vortexing or the intensity of shaking). However, the droplet volumes generated by such techniques continuously vary along the volume distribution produced.

As will be appreciated by one of ordinary skill in the art, the ranges for and volumes within a volume distribution will depend on a variety of factors for a given analysis. In some aspects, the volume distributions of the plurality of compartmentalized volumes can include a volume range from about 100 nanoliters (nL) to about 1 femtoliter (fL), from about 10 nL to about 10 fL, from about 1 nL to about 100 fL, from about 100 nL to about 1 picoliter (pL), from about 10 nL to about 10 pL, from about 1 nL to about 1 pL, from about 500 pL to about 50 fL, from about 100 pL to about 100 fL. Depending on the selected factors for producing compartmentalized volumes, it is routine to define the upper and lower boundaries of a volume distribution by, e.g., changing the intensity of mixing a sample and oil phase with a surfactant. There can be ranges of volumes in the volume distributions. For example, volumes in the distributions can range by more than a factor of 2, by more than a factor of 10, by more than a factor of 100, or by more than a factor of 1000. By ranging by a factor of 2, the lower boundary of the volume distribution can be, e.g., 10 nL with an upper boundary of 20 nL. Similarly, by ranging by a factor 10, the lower boundary of the volume distribution can be, e.g., 10 nL with an upper boundary of 100 nL.

In some aspects, the compartmentalized volumes have a volume from about 100 nanoliters (nL) to about 1 femtoliters (fL), from about 10 nL to about 10 fL, from about 1 nL to about 100 fL, from about 100 nL to about 1 pL, from about 10 nL to about 10 pL, from about 10 nL to about 100 pL, from about 10 nL to about 1 pL, from about 10 nL to about 10 pL, from about 1 nL to 1 about pL, from about 500 pL to about 50 fL, or from about 100 pL to about 100 fL. In further aspects, the compartmentalized volumes have a volume of 100 nanoliters (nL) to 1 femtoliters (fL), from 10 nL to 10 fL, from 1 nL to 100 fL, from 100 nL to 1 pL, from 10 nL to 10 pL, about 1 nL to 1 pL, from 500 pL to 50 fL, or from 100 pL to 100 fL.

Reactions (e.g., amplification) can be carried out in volumes with different sizes, before or during analysis of the volumes to determine in which volumes have undergone reaction (e.g., have amplified product). In certain examples, the volumes (e.g., compartmentalized volumes) can be sized and the number of occupied compartmentalized volumes (e.g., compartmentalized volumes containing a detectable agent) counted. All or just some of the compartmentalized volumes can be analyzed. Analysis can be achieved in a non-limiting example using droplets, by flowing the droplets in a single file through a flow cytometer or similar device, where the size of the droplet can be determined and the presence of amplification can be detected. The size of the droplet can, for example, determined based on the scattering signal from the droplet and the presence of amplification can be indicated by a fluorescence signal from the droplet. Alternatively, the diameter of compartmentalized volumes can be determined by microscopy. In a non-limiting example using droplets, the droplets can be extracted (before, during, or after completion of a reaction, e.g., amplification) from a sample holder and imaged in widefield with a CCD camera. The droplets, e.g., can be spread out on a surface, can be embedded between two glass slides, or can be in a microfluidic chamber and placed under a widefield microscope. By using appropriate excitation and emission filters the fluorescence within the droplet can be quantified to reveal the presence or absence of amplification. By noting both the size of the droplet and the presence or absence of amplification product in each droplet, it is possible to back-calculate the original concentration of the analyte present in the sample after interrogating a sufficient number of droplets of different sizes. Because the droplets are of different sizes, for a given dynamic range, the analysis is much faster than if the droplets are all of similar size. In some embodiments, the methods herein further include using a number of droplets in a plurality and the individual volumes of the droplets in the plurality to conduct digital measurements. For example, a sample concentration of a molecule of interest can be determined using the number of droplets in the plurality, the number of droplets in the plurality with one or more molecules of interest, and by measuring the volume of some or all of the droplets in the plurality. Example methods for determining sample concentrations can be found in the Examples section.

In certain embodiments, the detectable agent can be associated with a molecule of interest for detection. For example, the detectable agent can be associated with a nucleic acid molecule (e.g., DNA or RNA), a peptide, a protein, a protein conjugated to a nucleic acid, a nucleic acid conjugated to an antibody, a lipid, or other molecule (e.g., biomolecule) present in a sample. As defined herein, “associated” in the context of the detectable agent includes interaction with the molecule via covalent and/or non-covalent interactions. For example, the detectable agent can be covalently attached to the molecule of interest. Alternatively, the detectable agent can, for example, be an intercalation agent or a Taqman® probe that can be used to detect a nucleic acid molecule (e.g. a DNA and/or RNA molecule). Other detectable agents can be used, such as reference dyes that may not associate with molecules in a volume of interest.

Some embodiments of the present disclosure include producing compartmentalized volumes in contact with immiscible fluids. A wide variety of immiscible fluids can be combined to produce droplets of varying volumes. As described further herein, the fluids can be combined through a variety of ways, such as by emulsification. For example, aqueous solution (e.g., water) can be combined with an oil phase (e.g., polybutene) to produce droplets in a sample holder or on a microfluidic chip. Aqueous solutions suitable for use in the present disclosure can include a water-based solution that can further include buffers, salts, and other components generally known to be used in detection assays, such as PCR. Thus, aqueous solutions described herein can include, e.g., primers, nucleotides, and probes. Suitable oil phase compositions can comprise, but are not limited to, a polybutene, a mineral oil (e.g., light mineral oil), a silicone oil, a fluorinated oil or fluid (e.g., a fluorinated alcohol or Fluorinert), other commercially available materials (e.g., Tegosoft®), or a combination thereof.

In addition to aqueous solutions and non-aqueous fluids, surfactants can also be included to, e.g., improve stability of the droplets and/or to facilitate droplet formation. Suitable surfactants can include, but are not limited to, non-ionic surfactants, ionic surfactants, silicone-based surfactants, fluorinated surfactants or a combination thereof. Non-ionic surfactants can include, for example, sorbitan monostearate (Span 60), octylphenoxyethoxyethanol (Triton X-100), polyoxyethylenesorbitan monooleate (Tween 80) and sorbitan monooleate (Span 80). Silicone-based surfactants can include, for example, ABIL WE 09 surfactant. Other types of surfactants generally well known in the art can similarly be used. In some embodiments, the surfactant can be present at a variety of concentrations or ranges of concentrations, such as approximately 0.01%, 0.1%, 0.25%, 0.5%, 1%, 5%, or 10% by weight.

The present disclosure further includes determining a concentration of a sample. For example, the methods and systems can be used to determine (1) volumes of droplets and (2) a number of droplets that contain a detectable agent, which can be used to determine the concentration of a sample. This information can be used in a variety of ways to determine sample concentrations. For example, target molecules are present in the sample at a concentration in units of molecules/volume. The sample can be distributed into droplets of variable volumes that can be analyzed. The individual volumes of the droplets (all or just some) can be determined by methods provided herein. In addition, using detection methods described herein, droplets can be analyzed for containing a detectable agent or not. For a given sample concentration, some of the droplets may contain a detectable agent and some may not. For higher sample concentrations, generally more droplets of a plurality may contain detectable agents and vice versa; for low sample concentrations, fewer droplets of a plurality may be occupied by a detectable agent. As further described herein, the probabilities of occupancy by a detectable agent in a particular volume distribution can be defined for a wide range of sample concentrations, which can then be compared to real data to determine the concentration of an unknown sample.

Another aspect of the present disclosure comprises a device for carrying out the methods of the disclosure. Such devices may create arrays of digitized and discrete volumes of different sizes. In another embodiment, the device carries out the method for increasing the dynamic range of digital measurements of a sample, comprising creating a sample concentration gradient and creating sample volumes of different sizes.

In yet another aspect of the present disclosure, the methods, systems and devices described herein can be applied to isothermal amplification techniques, such as digital ELISA, NASBA, and LAMP. ELISA is protein based and usually used for the quantification of proteins or small molecules. NASBA and LAMP are isothermal amplification schemes that have been developed to complement PCR.

In an isothermal amplification, there is no temperature cycling occurring as in traditional PCR. There are several types of isothermal nucleic acid amplification methods such as transcription mediated amplification, nucleic acid sequence-based amplification, signal mediated amplification of RNA technology, strand displacement amplification, rolling circle amplification, loop-mediated isothermal amplification of DNA, isothermal multiple displacement amplification, helicase-dependent amplification, single primer isothermal amplification, and circular helicase-dependent amplification.

LAMP, which stands for Loop-Mediated Isothermal Amplification, is capable of amplifying DNA with high specificity, efficiency, and rapidity under isothermal conditions (˜60° C.). Because of the characteristics of its amplification reaction, LAMP is able to discriminate single nucleotide differences during amplification. As a result, LAMP has been applied for SNP (single nucleotide polymorphism) typing. LAMP has also been shown to have about 10 fold higher sensitivity then RT-PCR in the detection of viruses. Additionally, because LAMP amplification of DNA can be directly correlated with the production of magnesium pyrophosphate, which increases the turbidity of solution, the progress of LAMP has been monitored using a simple turbidimeter. Therefore, a non-homogenous assay can be used for detecting the amplification products that result from LAMP. The progress of LAMP reactions can also be measured using colorimetric assays.

In one aspect, the present disclosure provides a method for performing digital loop-mediated amplification of a sample. The method can include producing a plurality of compartmentalized volumes of the sample, wherein at least one compartmentalized volume in the plurality comprises a nucleic acid molecule (e.g., a DNA and/or a RNA molecule); and performing loop-mediated amplification in the at least one compartmentalized volume to produce amplified product of the nucleic acid molecule. The method can also include detecting the amplified product. In some embodiments, the method includes determining a number of compartmentalized volumes in the plurality that comprise the amplified product; and calculating a concentration of the nucleic acid molecule in the sample using individual volumes of the compartmentalized volumes in the plurality and the number of compartmentalized volumes in the plurality that contain the nucleic acid molecule. The microfluidic device can include a plurality of chambers configured to form the plurality of compartmentalized volumes.

Despite some of the advantages offered by NASBA and LAMP, one important drawback is the difficulty with performing quantification, which would be beneficial in most situations. Quantification often requires meticulous calibration and control using standards amplified under identical conditions, which can be very tedious (especially for field studies) and is not practical in many cases. For non-homogenous assays, such as the detection of precipitate in LAMP, accurate calibration can be especially challenging.

Rolling circle amplification (RCA) is an isothermal nucleic-acid amplification method. It differs from the polymerase chain reaction and other nucleic-acid amplification schemes in several respects. During RCA, a short DNA probe anneals to a target DNA of interest, such as the DNA of a pathogenic organism or a human gene containing a deleterious mutation. The probe then acts as a primer for a Rolling Circle Amplification reaction. The free end of the probe anneals to a small circular DNA template. A DNA polymerase is added to extend the primer. The DNA polymerase extends the primer continuously around the circular DNA template generating a long DNA product that consists of many repeated copies of the circle. By the end of the reaction, the polymerase generates many thousands of copies of the circular template, with the chain of copies tethered to the original target DNA. This allows for spatial resolution of target and rapid amplification of the signal. The use of forward and reverse primers can change the above linear amplification reaction into an exponential mode that can generate up to 1012 copies in 1 hour. The calibration required for such quantitative measurements can be cumbersome.

To overcome this drawback, the present disclosure provides digital isothermal amplifications, such as NASBA and LAMP, where the use of an array of digitized volumes, similar to digital PCR, is used for carrying out digital NASBA, digital LAMP, and rolling circle amplification. Furthermore, by using concentration gradients and/or arrays of digitized volumes of different sizes, we can effectively increase the dynamic range of these digital measurements. The current method ideally complements these isothermal amplification schemes to make them a quantitative technique for measuring the presence of RNA and DNA. In another embodiment of the present disclosure, the method is applied to antibody based amplification. In another embodiment, the method is applied to specific molecule recognition based amplification.

The particulars shown herein are by way of example and for purposes of illustrative discussion of the preferred embodiments of the present disclosure only and are presented in the cause of providing what is believed to be the most useful and readily understood description of the principles and conceptual aspects of various embodiments of the present disclosure. In this regard, no attempt is made to show structural details of the disclosure in more detail than is necessary for the fundamental understanding of the disclosure, the description taken with the drawings and/or examples making apparent to those skilled in the art how the several forms of the present disclosure can be embodied in practice.

Unless otherwise specified, the presently described methods and processes can be performed in any order. For example, a method describing steps (a), (b), and (c) can be performed with step (a) first, followed by step (b), and then step (c). Or, the method can be performed in a different order such as, for example, with step (b) first followed by step (c) and then step (a). Furthermore, those steps can be performed simultaneously or separately unless otherwise specified with particularity.

As used herein and unless otherwise indicated, the terms “a” and “an” are taken to mean “one,” “at least one” or “one or more.” Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to.” Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

Specific elements of any foregoing embodiments can be combined or substituted for elements in other embodiments. Furthermore, while advantages associated with certain embodiments of the disclosure have been described in the context of these embodiments, other embodiments can also exhibit such advantages, and not all embodiments need necessarily exhibit such advantages to fall within the scope of the disclosure.

While preferred embodiments of the present disclosure have been shown and described herein, it will be obvious to those skilled in the art that such embodiments are provided by way of example only. Numerous variations, changes, and substitutions will now occur to those skilled in the art without departing from the present disclosure. It should be understood that various alternatives to the embodiments of the present disclosure described herein can be employed in practicing the present disclosure. It is intended that the following claims define the scope of the present disclosure and that methods and structures within the scope of these claims and their equivalents be covered thereby.

### EXAMPLES

The specific dimensions of any of the apparatuses, devices, systems, and components thereof, of the present disclosure can be readily varied depending upon the intended application, as will be apparent to those of skill in the art in view of the disclosure herein. Moreover, it is understood that the examples and aspects described herein are for illustrative purposes only and that various modifications or changes in light thereof can be suggested to persons skilled in the art and are included within the spirit and purview of this application and scope of the appended claims. Numerous different combinations of aspects described herein are possible, and such combinations are considered part of the present disclosure. In addition, all features discussed in connection with any one aspect herein can be readily adapted for use in other aspects herein. The use of different terms or reference numerals for similar features in different aspects does not necessarily imply differences other than those expressly set forth. Accordingly, the present disclosure is intended to be described solely by reference to the appended claims, and not limited to the aspects disclosed herein.

### Example 1

### Digital PCR (dPCR) Amplification Comparison of Polybutene and Mineral Oil

This Example describes a comparison of digital PCR amplifications using polybutene and mineral oil.

TABLE 1
Phases used in PCR amplification thermal cycling.

| Phase | % oil | % Abil WE 09 | % Tegosoft ® DEC |
|---|---|---|---|
| Polybutene-10 | 33.00 | 0.02 | 66.98 |
| Polybutene-8 | 50.00 | 0.02 | 49.98 |
| Polybutene-6 | 67.00 | 0.02 | 32.98 |
| Mineral Oil | 67.00 | 0.02 | 32.98 |

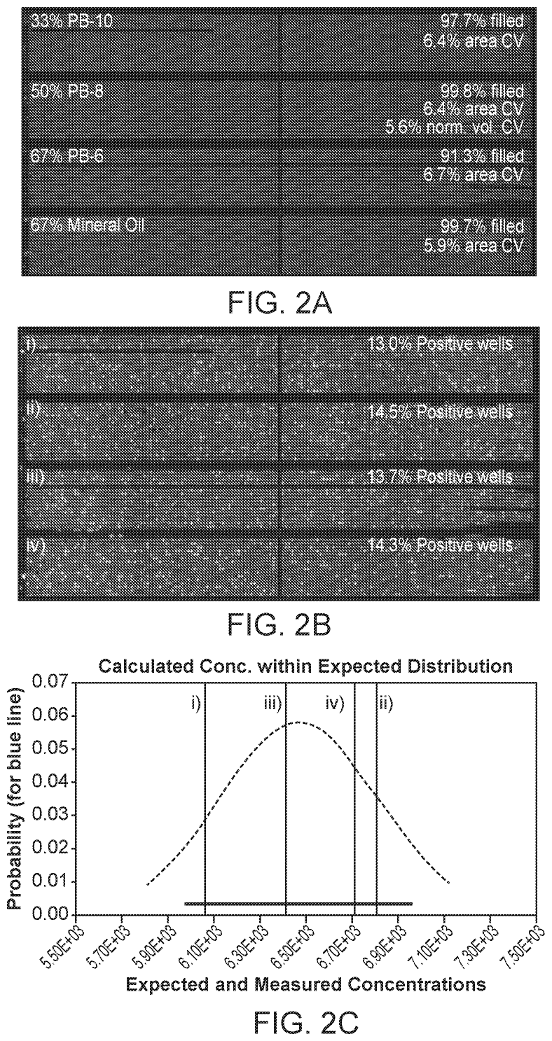

Thermal cycling was conducted using samples discretized in the various mineral oil or polybutene phases described in Table 1. As shown in FIG. 2A, samples were prepared using a SD chip platform, wherein each plate was first flushed with the oil phase, then loaded with an aqueous phase comprising 75 μL PCR solution from the following composition: 150 μL of 2× SsoFast™ Evagreen® Supermix, 30 μL of 20 mg/mL BSA solution, 15 μL of 5 μM Primer 1 solution, 15 μL of 5 μM Primer 2 solution, 15 μL of 10 mM Tris pH 8.0, 75 mM NaCL, 50 μM EDTA buffer solution, 75 μL of DNAse free water and 1 μL of a template solution. The loading of the aqueous phase was followed by a third fluid consisting of the originally used oil phase. The fluidic packets residing in fluidic harbors in contact with oil phase were thermally cycled to perform digital PCR. As seen in FIG. 2B, each individual packet was imaged during thermal cycling in order to perform digital PCR. Following thermal cycling, the arrays were analyzed and number of wells containing amplified product were determined. All arrays contained the same PCR mixture, and were therefore expected to give a similar percentage of wells containing amplified product. FIG. 2C shows that the number of positive wells was in agreement within the standard Poisson distribution confidence interval. All four samples were internally consistent with each other, and in agreement with the expected concentration. This result indicated that polybutene is compatible with the PCR reaction in the same manner in which mineral oil is.

### Example 2

### Effect of Polybutene and Mineral Oil on Substrates in Thermal Heating

This Example describes a comparison of the effects of thermal heating in the presence of polybutene and mineral oil on substrate materials typically used for digital PCR amplification.

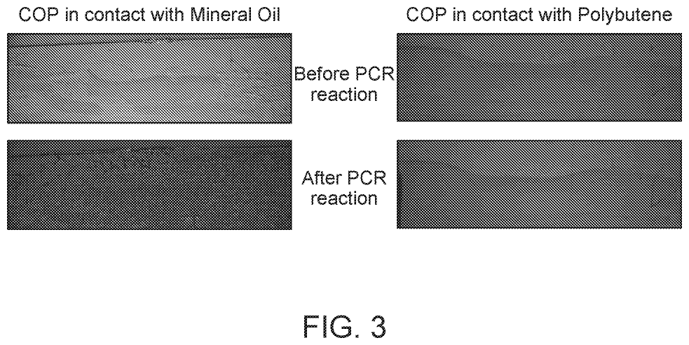

Cyclic olefin copolymer (COC) and cyclic olefin polymer (COP) are materials commonly used as substrates to make containers used for microfluidics, and possess low vapor permeability that should be compatible with dPCR applications. Mineral oil can degrade these plastics during thermal cycling. COP slides were thermalcycled under PCR conditions using either mineral oil or polybutene as a wetting layer between the PCR plate and the COP slide to improve thermal contact. Images were acquired prior to and following thermal cycling using a basic camera attached to a stereomicroscope. FIG. 3 shows that COP in contact with mineral oil under PCR thermal cycling conditions degraded, while COP in contact with polybutene under PCR thermal cycling conditions did not show noticeable degradation. This result indicates that polybutene is more compatible with materials used as PCR containers or chips than mineral oil under thermal cycling conditions. Polybutene should provide superior imaging qualities and improve device integrity during use in digital PCR or in digital isothermal amplifications.

### Example 3

### Broader Compatibility and Robustness Testing of Polybutene

This Example describes the effect of polybutene on digitization and fluorescence of different digital PCR solutions.

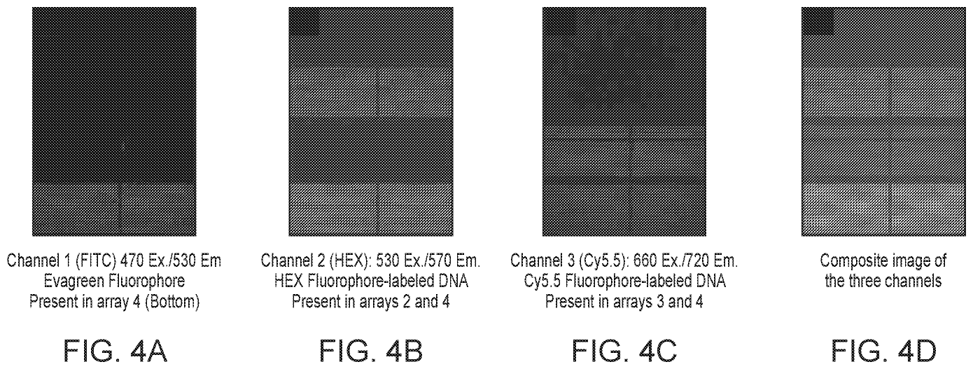

A testing of parallel digitization of different dPCR solutions and compatibility of different fluorescent probes under PCR conditions using a polybutene based oil phase was conducted. A polybutene phase of 67% polybutene-6, 0.02% Abil WE 09, and 32.98% Tegosoft® DEC was used. Arrays 1-3 contained SsoFast™ Probes Supermix, while Array 4 contained SsoFast™ Evagreen® Supermix. In FIG. 4, Array 1 is at the top and Array 4 is at the bottom. Array 1 contained no additional fluorophore. Array 2 contained 200 nM HEX™ fluorophore labeled DNA. Array 3 contained 200 nM Cy™5.5 fluorophore labeled DNA. For Array 4, besides the Evagreen® fluorophore inherently present, it also contained 200 nM each of HEX™ fluorophore labeled DNA and 200 nM Cy™5.5 fluorophore labeled DNA. As shown in FIG. 4, the digitization was successful and uniform across assay conditions, and all fluorophores tested were compatible with the polybutene phase. FIG. 4A shows Evagreen® fluorescence (470 Ex./530 Em.) in Array 4; FIG. 4B. shows HEX™ fluorescence (530 Ex./570 Em.) in Arrays 2 and 4; FIG. 4C shows Cy™5.5 fluorescence (660 Ex./720 Em.) in Arrays 3 and 4; FIG. 4D shows a composite image of all three channels. Each plate was analyzed using various emission bandwidths to selectively excite the probes. As can be seen in FIG. 4, all three fluorophores were found to be compatible with polybutene because their excitation and emission wavelengths were not noticeably affected by the oil and no other interference by polybutene were observed.

### Example 4

### Method for Generating Droplets of Variable Volumes Using Emulsion in a Tube

This Example provides exemplary methods for the production of polydisperse droplets of variable volume using an emulsion according to one aspect of the present disclosure. In this example, PCR tubes are used, however, any suitable vessel can be used according to the present disclosure.

An emulsion is prepared by introducing shear force to a mixture comprising an aqueous phase and a polybutene phase. The formation of an emulsion system is induced by vortexing individual tubes comprising the two phases. The aqueous phase containing the reaction mixture is pipetted into a 0.2 mL PCR tube that is prefilled with an appropriate polybutene-surfactant mixture.

The aqueous phase is pipetted into the polybutene mixture, and droplets of variable size are formed by vortexing for about 30 seconds. Emulsification is further enhanced by the addition of a small stir bar to the mixture, which promotes breakup of the aqueous phase into smaller droplets during vortexing. The presence of the surfactant in the polybutene stabilizes the emulsion, which reduces the frequency of droplet fusion in the mixture.

A system containing both aqueous and polybutene phases is added to a small collection of microtubes containing a small stainless steel bead. The tube is subsequently shaken at 15-17 Hz for 20 seconds to generate the emulsion. The emulsion is then transferred into a 0.2 mL PCR tube, and PCR is carried out in a Bio-Rad C1000 Thermal Cycler for 3 minutes at 95° C. (a hot start) and 50 cycles of 30 seconds at 95° C., 30 seconds at 54° C., and 30 seconds at 72° C. The emulsion is transferred to a multi-well plate by pipetting, and is imaged with a fluorescence microscope.

### Example 5

### Method for Generating Droplets of Variable Volume Using Emulsion in a Multi-Well Plate

This Example provides a method for the production of polydisperse droplets of variable volume using an emulsion, subsequent modification, and analysis according to one aspect of the present disclosure.

An optimized high-throughput process for droplet-emulsion PCR is developed. After a multichannel pipette loads polybutene phase and aqueous PCR reagents onto a multi-well plate, the entire multi-well plate is vortexed for 30 seconds to induce emulsification. The multi-well plate is subsequently fitted with a thermal cycler adaptor, and the mixture undergoes PCR amplification for 3 minutes at 95° C. (a hot start) and 50 cycles of 30 seconds at 95° C., 30 seconds at 54° C., and 30 seconds at 72° C. The multi-well plate is then imaged with a fluorescence microscope, with no further sample transfer steps required. In this example, any droplet instability (e.g. fusion) following emulsification will be independent of mechanical handling.

### Example 6

### Method for Generating Droplets Using Microfluidics

This Example provides exemplary methods for the production of droplets using microfluidics according to one aspect of the present disclosure.

Microfluidics is used to generate droplets of various sizes with a flow-focusing device. This uses specialized chips into which the two components (polybutene phase and aqueous sample) are first loaded. A microfluidic chip having a flow focusing channel geometry with two inlet reservoirs and one outlet reservoir is degassed. The polybutene phase is injected into one reservoir, and into the second reservoir is added an aqueous phase. Using polybutene as the continuous phase, and the aqueous mixture as the dispersed phase, flow focusing is used to generate aqueous droplets encapsulated by the polybutene phase. The monodisperse aqueous droplets suspended in polybutene are transferred into a 0.2 mL PCR tube, and PCR is carried out in a Bio-Rad C1000 Thermal Cycler for 3 minutes at 95° C. (a hot start) and 50 cycles of 30 seconds at 95° C., 30 seconds at 54° C., and 30 seconds at 72° C. The droplets in polybutene are transferred to a multi-well plate, and are imaged with a fluorescence microscope.

### Example 7

### Method for Generating Droplets of Monodisperse Volume Using Microfluidic Chip

This Example provides exemplary methods for the production of monodisperse droplets using microfluidics, subsequent modification, and analysis according to one aspect of the present disclosure.

An aqueous phase is prepared, and 20 μL is transferred into each of eight individual middle wells on a Bio-Rad DG8 cartridge. A polybutene mixture is prepared, and 70 μL is transferred into each of eight individual bottom wells of the cartridge. The cartridge is placed into a Bio-Rad QX200 Droplet Generator and processed to generate monodisperse droplets encompassed by the polybutene phase. Droplets are transferred to a 96-well plate and PCR amplification is conducted using 40 thermal cycles. The 96-well plate imaged with a fluorescence microscope.

### Example 8

### Sample Digitization Over a Wide Volume Range

This Example describes a device for digitizing samples over a wide volume range.

Self-digitization (SD) platforms are able to digitize samples over a wide volume range and array size. This enables handling large sample volumes for high sensitivity assays, large fluidic harbor numbers (small fluidic harbor volumes) for high resolution assays and intermediate fluidic harbor volumes and/or numbers for standard experimental applications. In one configuration, a design comprises of 640 fluidic harbors that are approximately 90 nL in volume. At a total volume of nearly 60 μL a detection limit of almost 50 molecules/mL is achieved. Fluidic harbors can be, e.g., 520 μm wide by 1000 μm long, with a 100 μm bevel and can be approximately 175 μm tall. The flow channel can be about 200 μm wide and about 40 μm tall. There can be, e.g., two drainage channels per fluidic harbor and three junctions per channel to better facilitate polybutene drainage. Drainage channel junctions can be approximately 8 μm wide. In some aspects, the drainage channel widens as junctions merge, and splits as it meets the flow channel to maintain a minimal cross section to prevent aqueous sample of travelling through the drainage channel. Devices can be filled in approximately 5 minutes.

Another configuration includes a design comprising of 1024 fluidic harbors that can be approximately 7.5 nL in volume. The total sample volume needed (approximately 8 μL) is comparable to a reasonable minimal sample volume for many assays. Fluidic harbors can be, e.g., 200 μm wide×400 μm long with a 50 μm bevel and can be approximately 100 μm tall. The flow channel can be 80 μm wide, approximately 25 μm tall and feature small indents (approximately 20 μm×approximately 30 μm) on both sides to help direct fluid flow and encourage stable break-off of the aqueous sample (second fluid) when the final polybutene stream (third fluid) comes through to digitize the sample. A pair of drainage channels (approximately 8 μm wide) with two junctions per channel can be used for each fluidic harbor. This provides rapid enough polybutene drainage to produce nearly stepwise sample loading. Loading can be achieved, e.g., in <2 minutes. Devices are filled with a coefficient of variation (CV) of approximately 3% or less.

Another configuration includes a design including >10,000 fluidic harbors which are approximately 1 nL in volume. With this number of fluidic harbors samples that are different in concentration by a factor of approximately 1.02-1.06 (resolution limit) can be achieved with a confidence level of at least 95% at a 95% power level. Designs for 25,600 fluidic harbors that are 80 μm×160 μm with 20 μm bevel and 80 μm tall, and 10,240 fluidic harbors that are 100 μm×200 μm with 25 μm bevel and 80 μm tall are filled and digitized. The flow channel is 50 μm wide and approximately 20 μm tall. The drainage channel is simpler because less volume must be displaced, with two sets of single junction drainage channels (approximately 8 μm wide) per fluidic harbor. Devices are filled and digitized in <5 minutes.

Another configuration includes a design comprising of 10,240 fluidic harbors which are approximately 150 pL in volume. With this number of fluidic harbors samples that are different in concentration by a factor of approximately 1.05-1.09 (resolution limit) can be achieved with a confidence level of at least 95% at a 95% power level. Designs for 10,240 fluidic harbors that are 50 μm×80 μm with 10 μm bevel and approximately 40 μm tall are filled and digitized. The flow channel is approximately 25 μm wide and approximately 17 μm tall. The drainage channel is simpler because less volume is displaced, with one single junction drainage channel (approximately 8 μm wide) per fluidic harbor. Devices are filled and digitized in <5 minutes.

Another configuration includes a design comprising of 10,240 fluidic harbors which are approximately 50 pL in volume. With this number of fluidic harbors samples that are different in concentration by a factor of approximately 1.05-1.09 (resolution limit) can be achieved with a confidence level of at least 95% at a 95% power level. Designs for 10,240 fluidic harbors that are 50 μm×30 μm with 10 μm bevel and approximately 40 μm tall are filled and digitized. The flow channel is approximately 25 μm wide and approximately 17 μm tall. The drainage channel is simpler because less volume must be displaced, with one single junction drainage channel (approximately 8 μm wide) per fluidic harbor. Devices are filled and digitized in <5 minutes.

All of these configurations function with multiple grades and blends of polybutene including polybutene grades from 300 Mn-2,400 Mn and blends comprising an isobutylene content between 10% and 100%. One embodiment of the polybutene system can consist of Abil WE 09 as a surfactant, Tegosoft® DEC as a wetting agent and also to lower viscosity, and can also include hexadecane to further lower viscosity. Abil WE 09 can be used at approximately 0.02% volume/volume. Under some conditions, if Abil concentrations are too high it can result in droplets (that can be generated during flow) to not coalesce. Droplet formation can form either at the inlet or at the flow channel/fluidic harbor junction. Droplet formation at the flow channel/fluidic harbor junction is more prominent in larger fluidic harbors as the increased polybutene drainage creates the dual aqueous:polybutene flow that can lead to droplet formation. Tegosoft® can be used over a wide range (approximately 3.5-approximately 83%), but is not restricted to this range. Hexadecane can be used in some aspects to lower viscosity and can be used at approximately 30%, but hexadecane can cause swelling of PDMS.

Some parameters for successful digitization include the wetability of the device by the polybutene blend, the polybutene/aqueous surface tension, the flow rate, the polybutene viscosity, and the device dimensions. In the 32.98% Tegosoft® DEC, 67.00% polybutene, and 0.02% ABIL WE 09 system, the Tegosoft® provides outstanding wetability of the polybutene to the device, but if the viscosity is too high drainage can slow resulting in slower filling of individual fluidic harbors and greater droplet generation resulting in less stepwise filling.

### Example 9

### Digital PCR Amplification Using Polybutene and Analysis

This Example describes a method for dPCR amplification and analysis of a nucleotide sample using a droplet emulsion system in polybutene. Polybutene is used in the formation of discretized samples and in thermal cycling.

In order to visualize the presence of amplification products within a droplet, a fluorescence probe is added to the reaction mixture that specifically recognizes the presence of the amplicon. A small amount (1-2 μM) of the red fluorescent dye 6-carboxy-X-rhodamine (ROX) is used as a reference dye in the reaction mix. The spectral signature of ROX™ is readily distinguished from that of a green fluorescence probe that is used to report amplification. Furthermore, the ROX™ fluorescent signal is insensitive to amplification or other reaction or reagent conditions. The ratio of two intensities, one measured from the reference dye and one measured from the probe, is used to make a binary measurement from each measured droplet. The intensity ratio is not affected by changes in droplet volume due to fusion or shrinkage, or unwanted changes in light excitation power, since each of these will affect fluorescence intensity of both dyes comparably, leaving the ratio unchanged.

Droplets of aqueous solution suspended in polybutene are generated using an emulsion technique. In order to build a profile of the distribution of droplet sizes, the emulsion is transferred onto a 96-well plate, the surface of which is silanized, and covered with an excess volume of the polybutene mixture. Emulsions are imaged using a Zeiss LSM 510 confocal microscope in multi-track mode with a PLAN APO 20X, 0.75 NA objective. Laser-source excitation wavelengths of 543 nm (LP 610) and 488 nm (BP 500-530) are used to collect fluorescence signals from the ROX™ and FAM™ dyes. An additional step is verifying the presence of PCR amplification products in droplets during data acquisition. The concentration of amplified DNA sample is determined using the intensity ratio of the green and red fluorescent dyes, together with the number of positive and negative droplets.

### Example 10

### dLAMP Amplification Using Polybutene

This Example describes a method of using polybutene for dLAMP amplification.

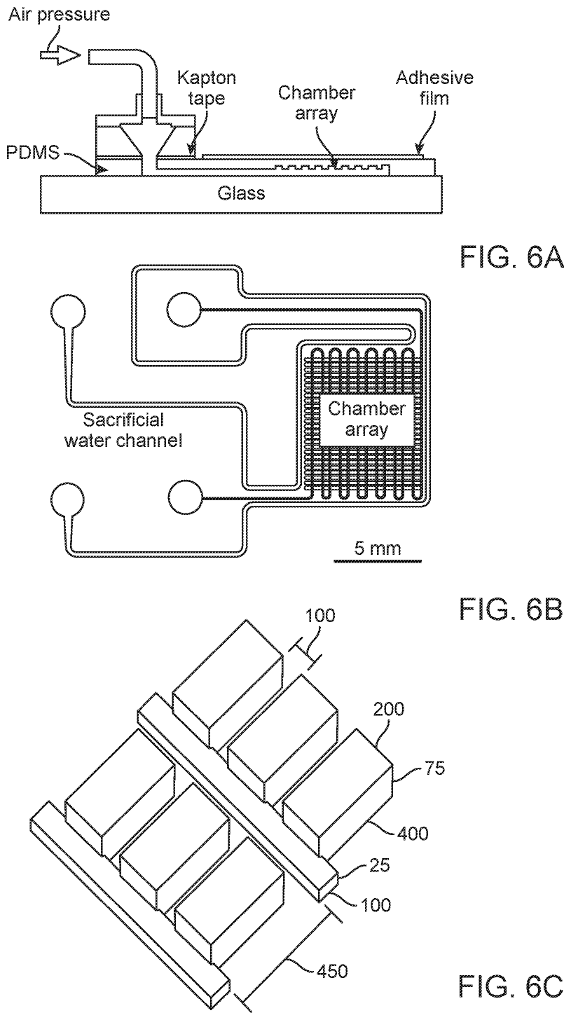

The digital LAMP chip is designed as a series of rectangular cavities to hold the droplets, which are positioned along a smaller rectangular channel used for sample delivery (FIGS. 6A-C). The chip layout is designed to improve droplet stability and retention, automation of the filling process, and operation at elevated temperatures. A reduction of the height ratio between main channel and side cavities to ⅓ reduces the chance of crosstalk between chambers. The depth of the side chamber is extended to 400 μm, which improves droplet retention. Air pressure regulated flow is used to ensure reproducibility and robustness of the automated filling. Evaporation of the aqueous droplets at higher temperatures is minimised by various measures: a) The chambers are arranged in a dense array and embedded between only a thin top and bottom layer of PDMS; b) an additional sacrificial water channel is placed around the array; c) a self-adhesive film is added on top of the chip as a vapour barrier; d) a solution comprising polybutene is used as the continuous oil phase.

Prior to loading the aqueous phase, the chip is primed with the continuous phase (32.98% Tegosoft® DEC, 67.00% polybutene PB-6, and 0.02% ABIL WE 09 surfactant). Fresh LAMP solution is introduced into the inlets and self-digitized into nanoliter-sized droplets inside the side chambers. The aqueous phase enters the main channel and slowly displaces the polybutene from the side chambers. Once the whole sample enters the chip, the tailing polybutene phase shears off the fluids at the opening of the side compartments and isolates the nanoliter droplets.

### Example 11

### Centrifugal Loading

This Example describes a method of loading or filling a device with a fluid using centrifugal force.

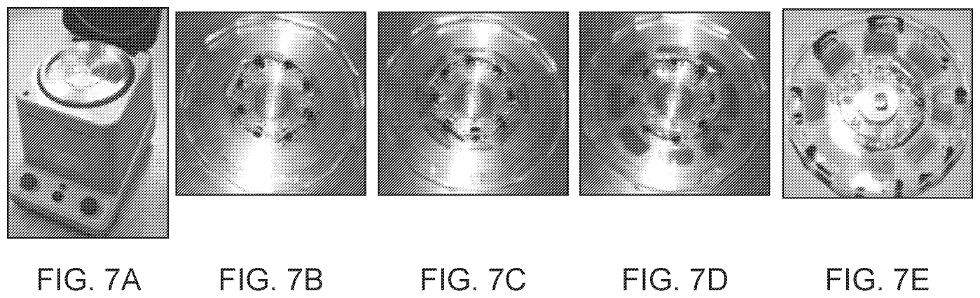

Loading and digitization of SD devices can use the following protocol in some example experiments. The polybutene system is 0.02% Abil, 33% Tegosoft® and 67% polybutene PB-6. Fluorocarbon can also be used. In this configuration SD devices are spun using a bench-top centrifuge with a custom rotor designed for discs, as shown e.g., in FIG. 7A. In another configuration other rotary mechanisms including optical disc drives are used. And in another configuration multiple rectangular SD chips are loaded onto a large centrifugal rotor (see, e.g., FIG. 8). PDMS devices are degassed in a vacuum desiccator for >5 minutes; other plastic materials besides PDMS can also be used. Then the polybutene system (approximately 20-30 μL) is loaded into each inlet reservoir and the disc is spun at approximately 1250 RPM for 1 minute. Then aqueous sample (approximately 10 μL) is loaded and the remaining reservoir filled with the polybutene system. Devices with the outlet at the outer portion of the disc are filled by spinning at approximately 1250 RPM for 2 minutes. Devices with the outlet at the inner portion of the disc are filled by spinning at approximately 1250 RPM for 4 minutes. Once filled these devices are stable under speeds up to approximately 3000 RPM. Filled arrays can be seen, e.g., in FIGS. 7B and 7C, and a filled device comprising of several arrays is shown in FIG. 8. A time series during filling can be seen in FIGS. 7B-7E.

### Example 12

### Loading of Linear Centrifuge Device

This example describes loading of a linear centrifuge device. The loading instrument includes a stepper motor as the rotary actuator, capable of 1600 microsteps/rotation and >1200 rotations per minute. Attached to the rotary actuator is the rotor assembly containing two receptacles for securing the microfluidic devices. The rotary assembly has a housing that also anchors the rotary actuator to enable safe rotation of the assembly and devices. The actuator is controlled through a computer on software provided by the manufacturer and/or distributor of the rotary actuator.

Each device could contain up to 8 arrays, with each array comprised of 2560 fluidic compartments, and each compartment comprising a volume of approximately 23 nL. The devices consist of a 3″×4″ piece of glass that is spin-coated with PDMS. Bonded to this is another piece of PDMS containing the device features. This piece of PDMS consists of three regions. The central region is thin (between 300-1000 μm thick) and spanned the entire area encompassing the fluidic compartments. The proximal region is thick (3-10 mm thick) and contains the inlets, inlet reservoirs, outlets and part of the channels that are in fluidic communication with the fluidic compartments. The distal region is also thick (2-10 mm thick) and contains outlet reservoirs and portions of the channels that allowed for fluidic communication with the fluidic compartments in the central region and the outlets in the proximal region. Over the central region a vapor barrier is bonded. In some embodiments this is glass, in other embodiments it is PCTFE, in other embodiments it is some other vapor barrier material. In some embodiments the vapor barrier is plasma sealed to the PDMS. In other embodiments an adhesive layer is used.

In one embodiment the typical oil used to preload the device is 0.02% Abil WE 09, 33% Tegosoft® DEC and 67% polybutene. The device is degassed in a vacuum desiccator then the oil is loaded into the inlet reservoir and the device centrifuged in the rotor assembly at ˜900-1200 RPM for 2-10 minutes. In some embodiments it is necessary to load additional oil into the inlet reservoir and additional rounds of centrifuging are performed. In some embodiments the partly loaded device is placed in the vacuum desiccator under low vacuum pressure to continue to evacuate air bubbles. Once all air bubbles are removed from the wells the device can be loaded with sample.

Sample loading occurs by adding sample (˜60-80 μL for this design) to the inlet reservoir through the inlet with a pipette. Once sample is loaded the device is loaded and secured onto the rotor and the rotor centrifuged at 900-1200 RPM for 4-10 minutes, or until all the sample has settled in the fluidic compartments or outlet reservoir.

### Example 13

### Amplification Reactions with Polybutene in a Cyclic Olefin Polymer (COP) Device

This Example describes the effect of polybutene on digitization, fluorescence, and amplification, and compatibility of various digital PCR solutions when used in a COP device, as well as a glass-backed PDMS device.

An oil sample was prepared containing 0.02-0.08% Abil WE 09, 60-70% Tegosoft DEC and 30-40% PB-6. This oil sample was used to prime two devices. Device 1 was composed of glass-backed PDMS, and Device 2 was composed of COP plastic. Four separate aqueous samples were prepared, all containing Ssofast™ Evagreen® supermix. Samples 1 and 2 contained a relatively low concentration of a purified synthetic DNA sample that should generate positive signals in about 1% of compartments. Samples 3 and 4 contained a relatively high concentration of purified synthetic DNA sample that should generate positive signals in about 95% of compartments. Samples 2 and 4 also contained a TYE™-665 reference dye.

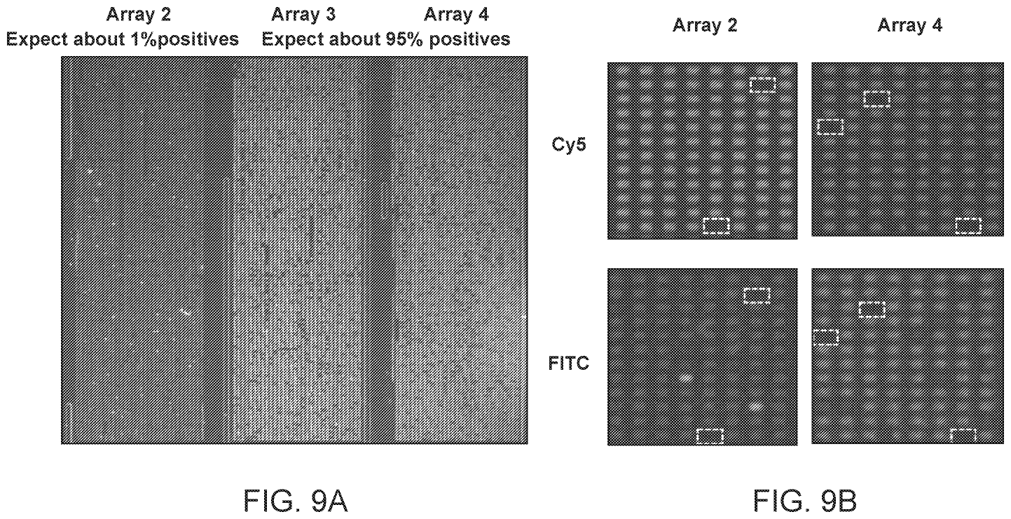

Samples 1-4 were loaded into arrays 1-4 respectively. Device 1 was loaded and digitized using centrifugal filling for a total of 8 minutes at 900 RPM. Device 2 was loaded by using vacuum pressure applied to the outlet, and each sample was pulled through until additional oil came through to cap off compartments and displace sample from the channels. Fluorescent images in both the “FITC” and CY™5” channel were acquired before amplification, then the devices were placed on “in-situ” adapters on an Eppendorf Mastercycler and a PCR reaction cycle was run. After amplification the devices were imaged again. FIG. 9A shows the fluorescent image of the FITC channel after amplification, showing sections of arrays 2-4. Array 2 contained a relatively low concentration of target DNA and was expected to produce about 1% positive wells; arrays 3 and 4 contained a much higher relative concentration and were expected to produce about 95% positive wells.

FIG. 9B shows the fluorescent image of the Cy™5 and FITC channels after amplification for sections of arrays 2 and 4. As shown in FIG. 9B, the reference dye (TYE™-665) in the Cy™5 channel can differentiate between empty wells and filled wells, while the amplification signal from Evagreen® in the FITC channel can differentiate between positive wells and negative wells. Accordingly, differentiation can be made between empty wells and negative wells, particularly in the FITC channel of Array 4. The dashed white boxes of FIG. 9B show empty wells to aid in the visibility of the empty wells. There was a clear identification of positive compartments based on increased fluorescence intensity of some compartments and a clear bimodal distribution of intensity. The observed fraction of positive wells was similar between devices and with the estimated expected rate.

The results show that a COP device can be used in the digitization of samples using polybutene. Amplification reactions occurred in the presence of polybutene in the COP device, as evidenced by the observed fluorescence. The samples with polybutene were compatible with the COP device.

### Example 14

### Measuring Concentration of Samples Using Polybutene in a Cyclic Olefin Polymer (COP) Device

This Example describes polybutene used in combination with a device composed of COP, and describes the amplification of solutions used to determine the original concentration of said solutions.

An oil sample was prepared containing 0.02-0.08% Abil WE 09, 60-70% Tegosoft DEC and 30-40% PB-6. This oil was used to prime a device composed of COP plastic. Aqueous samples containing Taqman DNA Polymerase, Reverse transcriptase and extracted RNA containing BCR-ABL sequences, and a Taqman probe containing a Cy™5 family dye were prepared. Sample 1 had a nominal concentration of about 40×10⁶ copies/mL. Sample 2 had a 1000-fold lower concentration of the target RNA, that is, about 40×10³ copies/mL.

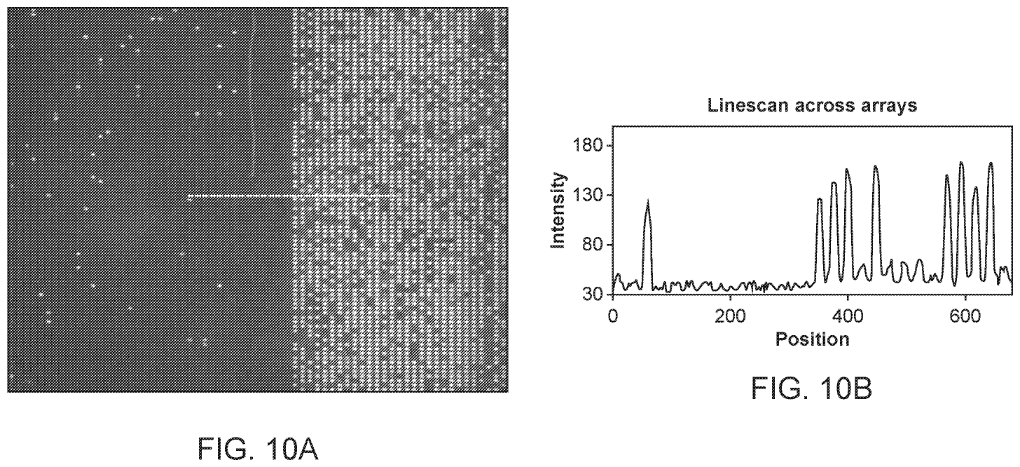

The samples were directed into the COP device using a vacuum pressure applied to the outlet, and each sample was pulled through until additional oil came through, thus capping off compartments and displacing sample from the channels. The devices were imaged. The COP device was placed on an “in-situ” adapter on an Eppendorf Mastercycler and an RT-PCR reaction cycle was run. After amplification the devices were imaged again (FIG. 10A). FIG. 10A shows the fluorescence of a section of the COP device using the Cy™5 channel after amplification. The left array shows Sample 2, which was expected to have a 1000-fold lower concentration of the target BCR-ABL mRNA in comparison to Sample 1 (the right array). The dashed line in FIG. 10A represents where a linescan (corresponding to FIG. 10B) was taken to identify positive and negative compartments. FIG. 10B shows the linescan across the two arrays, and shows the difference in intensity between the background fluorescence, negative compartments, and positive compartments.

There was a clear identification of positive compartments based on the substantial fluorescence intensity of some compartments and a clear bimodal distribution of intensity. From a subset of the device, an array that contained the low concentration (Sample 2) showed approximately 45 positive compartments out of approximately 2604 total compartments, which corresponds to an estimated concentration of 29,000 copies/mL, with a 95% CI from 20,500 to 37,400. From a subset of the device, an array that contained the high concentration (Sample 1) showed approximately 1769 positive compartments out of approximately 2436 total compartments, which corresponds to an estimated concentration of 21,500,000 copies/mL, with a 95% CI from 20,400,000 to 22,600,000. Accounting for the dilution factor, these results are in statistical agreement with each other and the absolute concentration is similar to the roughly estimated initial concentration of about 40,000 and 40,000,000 copies/mL.

The results show that a COP device can be used in amplification of samples using polybutene. Amplification reactions occurred in the presence of polybutene in the COP device, as evidenced by the observed fluorescence. The intensity of the fluorescence could be measured in the device, and the concentration of the samples could be determined. The samples comprising polybutene were compatible with the COP device.

1. A method for performing a digital assay, the method comprising:
producing a plurality of compartmentalized volumes, wherein:
each compartmentalized volume comprises an aqueous solution;
at least some of the compartmentalized volumes comprise a nucleic acid; and
at least some of the compartmentalized volumes are contacted with an oil phase comprising polybutene;
amplifying the nucleic acid to produce an amplified nucleic acid product; and
analyzing at least some of the plurality of compartmentalized volumes to determine the presence or absence of the amplified nucleic acid product.

2. The method of claim 1, further comprising:
determining the volume of at least some of the compartmentalized volumes; and
determining the concentration of the nucleic acid in the sample based on:
the presence or absence of the amplified nucleic acid product in the compartmentalized volumes; and
the volumes of the at least some compartmentalized volumes.

3. The method of claim 1, wherein the plurality of compartmentalized volumes are in the form of a plurality of droplets.

4-5. (canceled)

6. The method of claim 1, wherein the plurality of compartmentalized volumes are provided as an emulsion of the aqueous solution and the oil phase.

7-10. (canceled)

11. The method of claim 1, wherein the plurality of compartmentalized volumes are positioned in a microfluidic device.

12. The method of claim 11, further comprising loading the microfluidic device in sequence with:
a first oil phase;
an aqueous solution; and
a second oil phase comprising polybutene,
such that after loading:
at least a portion of the plurality of fluidic harbors comprises the aqueous solution separated into the compartmentalized volumes and separated by the second oil phase.

13. The method of claim 12, wherein the first oil phase comprises polybutene.

14. (canceled)

15. The method of claim 11, wherein the device comprises cyclic olefin polymers.

16-26. (canceled)

27. The method of claim 1, wherein the aqueous solution further comprises a detectable agent for labeling the at least one nucleic acid sequence.

28-30. (canceled)

31. The method of claim 1, wherein the amplifying comprises applying to the at least one compartmentalized volumes at least one temperature, wherein the at least one temperature is sufficient for amplification of the nucleic acid, wherein an isothermal temperature is applied to the device during the amplifying.

32. The method of claim 1, wherein a variable temperature is applied to the device during the amplifying

33. (canceled)

34. The method of claim 1, wherein the amplifying comprises polymerase chain reaction (PCR), rolling circle amplification (RCA), nucleic acid sequence based amplification (NASBA), loop-mediated amplification (LAMP), Recombinase Polymerase Amplification (RPA), Helicase-Dependent Amplification (HDA), or a combination thereof.

35-36. (canceled)

37. The method of claim 1, wherein the compartmentalized volumes have a volume from at least 1 femtoliters (fL) to not more than 100 nanoliters (nL), from at least 10 fL to not more than 10 nL, from at least 100 fL to not more than 1 nL, from at least 1 picoliters (pL) to not more than 100 nL, from at least 10 pL to not more than 10 nL, from at least 100 pL to not more than 10 nL, from at least 1 pL to not more than 10 nL, from at least 1 pL to not more than 1 nL, from at least 50 fL to not more than 500 pL, or from at least 100 fL to not more than 100 pL.

38. (canceled)

39. The method of claim 1, wherein the amplifying of the nucleic acid is compatible with polybutene, and wherein being compatible with polybutene comprises an ability to detect the amplified nucleic acid product.

40-43. (canceled)

44. The method of claim 1, wherein at least some of the compartmentalized volumes comprise a nucleic acid and a protein.

45. The method of claim 44, wherein the nucleic acid is conjugated to the protein.

46. The method of claim 1, wherein at least some of the compartmentalized volumes comprise a nucleic acid and an antibody, and wherein the nucleic acid is conjugated to the antibody.

47. (canceled)

48. The method of claim 46, wherein the at least some of the compartmentalized volumes comprise a target protein.

49-58. (canceled)

59. A microfluidic device for discretizing a fluidic sample, the device comprising:
an inlet port;
at least one flow channel having a flow axis, the at least one flow channel in fluidic communication with the inlet port;
a plurality of compartmentalized volumes in fluidic communication with the at least one flow channel and offset from the flow axis, wherein at least some of the plurality of compartmentalized volumes are contacted with an oil phase comprising polybutene; and
an outlet port in fluidic communication with the flow channel.

60-63. (canceled)

64. A method of introducing a fluid into a microfluidic device, the method comprising:
obtaining the microfluidic device of claim 59; and
introducing a first fluid into the flow channel of the microfluidic device, wherein the first fluid comprises polybutene.

65-72. (canceled)
